(19) United States
(12) Patent Application Publication
(10) Pub. No.: US 2025/0288790 A1
(43) Pub. Date: Sep. 18, 2025

(54) LAMPREY LOCK DEVICE

(71) Applicant: THE REGENTS OF THE UNIVERSITY OF CALIFORNIA, Oakland, CA (US)

(72) Inventors: Lucas R. Cusumano, Los Angeles, CA (US); Justin P. McWilliams, Los Angeles, CA (US)

(21) Appl. No.: 19/222,107

(22) Filed: May 29, 2025

Related U.S. Application Data
(63) Continuation-in-part of application No. 18/472,670, filed on Sep. 22, 2023.
Application No. 18/472,670 is a continuation of application No. PCT/US22/21379, filed on Mar. 22, 2022.
(60) Provisional application No. 63/164,123, filed on Mar. 22, 2021.

(51) Int. Cl. A61M 39/22

(52) U.S. Cl. CPC A61M 39/22; A61M 2039/229

(57) ABSTRACT

The present invention provides a lamprey lock device configured to provide a fluid transfer between two devices or objects. In one embodiment, lamprey lock device of the present invention improves fluid transfer by maximizing the inner diameter of connections between two objects including but not limited to a catheter, tubing, veress needles, trocars, syringes, or gas/fluid delivery systems.

### CROSS-REFERENCE TO RELATED APPLICATIONS

This application is a continuation-in part-of and claims priority to U.S. patent application Ser. No. 18/472,670 filed Sep. 22, 2023, which claims priority to International Application No. PCT/US22/21379 filed Mar. 22, 2022, which claims priority to U.S. Provisional Patent Application No. 63/164,123 filed Mar. 22, 2021, the contents of which are each incorporated by reference herein in their entirety.

### BACKGROUND OF THE INVENTION

Connections between catheters/tubing are necessary for a wide range of medical procedures including but not limited to drainage of intracorporal fluid collections, intravenous access and etc. to form a secure, leak-free connection for the transfer of fluids or gasses between the two devices or objects.

Larger diameter catheters have the advantage of more rapid flow of fluids. However, flow rates can be limited at connections due to the smaller diameter of the connection compared to the remainder of the catheter.

Thus, there is a need in the art for a connector device that allows connection of catheters/tubing with a larger inner diameter when compared to the widely utilized luer lock connection to minimize flow disturbance. The present invention meets this need.

### SUMMARY OF THE INVENTION

In one aspect, the present invention provides a lamprey lock device comprising: a tubular shaped housing having a proximal end, a distal end and length therebetween, the housing further having an external surface and an internal surface, such that a lumen is formed within the internal surface along its length; an O-ring positioned within the lumen at the distal end; a first luer taper adapter positioned within the lumen and proximal to the O-ring, wherein the first luer taper adapter has a threaded internal surface; and an elastomeric ring positioned within the lumen and proximal to the first luer taper adapter. In one embodiment, the O-ring, the first luer taper adapter and the elastomeric ring each has an internal diameter equal to or greater than 4 mm. In one embodiment, the lamprey lock device further comprising a second luer taper adapter positioned within the lumen and proximal to the elastomeric ring, wherein the second luer taper adapter has a threaded internal surface. In one embodiment, the elastomeric ring has a width of greater than 1 mm. In one embodiment, the lamprey lock device further comprising an elastomeric coating on the internal surface of the first luer taper adapter. In one embodiment, the threaded internal surface of the first luer taper adapter has a spiral rotation of at least 0.5. In one embodiment, the elastomeric ring has an interior diameter ranging between 5-8 mm. In one embodiment, the device further comprises a three-way stopcock comprising: a tubular body having a first tube section, a second tube section and a third tube section, wherein each of the first, second and third tube sections comprises a lumen and are fluidly connected at a first end to a central connector and terminate in an opening at a second end opposite to the first end; and a central component comprising a circumference that is slightly less than a circumference of the central connector, such that central component may be inserted within the central connector with minimal tolerance, and wherein the central component comprises a first open side, a second open side, a third open side, and a closed side; wherein the second end of the first tube section is connected to the distal end of the lamprey lock device; wherein the second end of the second tube section is fluidly connected to a flush pump; and wherein the second end of the third tube section is fluidly connected to a drainage reservoir. In one embodiment, the stopcock comprises: a drain mode, wherein the central component produces a patent channel from the first open side and the third open side into a drainage reservoir; and a flush mode, wherein the central component produces a patent channel from a flush reservoir through the first open side and the second open side into a catheter connected to the lamprey lock device. In one embodiment, the device further comprises a tubing having a proximal end, a distal end, and a body therebetween connected to the distal end of the housing, wherein the body comprises a first opening at the proximal end and a second opening at the distal end, a third opening therebetween and a side port, wherein the side port is fluidly connected to the body through the third opening. In one embodiment, the side port comprises a first end, a second end and a lumen therebetween, wherein the second end comprises an opening, sized and configured to connect to standard sizing for luer connectors. In one embodiment, the side port further comprises a valve member configured to allow selective fluid communication between the body and the side port, wherein the valve member comprises a compressible component configured to receive the luer tip of a syringe at the second end, a support structure configured to guide the compressible component through its travel along the lumen from the second end to the first end and at least one channel configured to open when the compressible component receives the luer tip of a syringe. In one embodiment, the device further comprises a flow switch positioned on the body and distal to the side port. In one embodiment, the flow switch comprises a tab, a track configured to allow longitudinal movement of the tab between an open and close position, a compressible tubing positioned, and an occluding member configured to move vertically pushing on the compressible tubing causing flow restriction between the body and a drainage reservoir. In one embodiment, the device further comprises an external clamp positioned distal to the side port and around the body. In one embodiment, the external clamp comprises a rounded opening and an occluded slot, wherein the rounded opening converges to create a narrow width of the occluded slot, and wherein when body is forced into the occluding slot, the side wall of the body is pinched together and prevents the flow of fluid therethrough. In one embodiment, the device further comprises a chamber positioned anywhere between the first opening and the second opening and is configured to engage the exterior surface of the body. In one embodiment, the chamber further comprises a first end, a second end, and a side port positioned at the first end and a bottom part positioned at the second end, and wherein the side port is fluidly connected to the body through a side channel. In one embodiment, the side port comprises a valve member configured to allow selective fluid communication between the body and the side port, wherein the valve member comprises a compressible component configured to receive the luer tip of syringe and a tip positioned below the compressible component, wherein once the syringe is inserted, the luer tip pushes the compressible component down on the body all the way to the bottom part, which in turn causes the side channel to open and allow fluid communication between the side port and the body. In one embodiment, the tip is a pointed tapered tip of at least 45 degrees.

In one aspect, the present invention provides a method of draining a body cavity comprising the steps of: providing a lamprey lock device comprising a tubular shaped housing having a proximal end, a distal end and length therebetween, the housing further having an external surface and an internal surface, such that a lumen is formed within the internal surface along its length; an O-ring positioned within the lumen at the distal end; a first luer taper adapter positioned within the lumen and proximal to the O-ring, wherein the first luer taper adapter has a threaded internal surface; and an elastomeric ring positioned within the lumen and proximal to the first luer taper adapter; inserting a catheter into the proximal end of the lamprey lock device, engaging the plurality of threads with clockwise rotation; placing a tubing at the distal end of the lamprey lock device comprising a proximal end, a distal end and a body therebetween; passing through a liquid through the catheter and the tubing.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 depicts a side view of an exemplary lamprey lock device of the present invention.

FIG. 2 depicts a sagittal cross section of an exemplary lamprey lock device of the present invention wherein the lamprey lock device is made of a rigid plastic material.

FIG. 3 depicts a sagittal cross section of an exemplary lamprey lock device of the present invention wherein the inner surface of the lumen of the lamprey lock device is covered with an elastomeric material.

FIG. 4 depicts a sagittal cross section of an exemplary lamprey lock device of the present invention wherein the lamprey lock device is made of a rigid plastic core and an elastomeric material covering the inner surface of the lumen.

FIG. 5 depicts a side view of a catheter connection with the lamprey lock device.

FIG. 6 depicts a coronal view of a catheter connection with the lamprey lock device.

FIG. 7 depicts a side view of an exemplary lamprey lock device of the present invention comprising a circumferential elastomeric seal.

FIG. 8 depicts a side view of an exemplary lamprey lock device of the present invention comprising a second luer taper positioned proximal to the circumferential elastomeric seal.

FIG. 9 depicts a side view of an exemplary lamprey lock device of the present invention connected to a drainage catheter.

FIG. 10 depicts a side view of an exemplary lamprey lock device of the present invention comprising an external clamp in a closed position.

FIG. 11 depicts a side view of an exemplary lamprey lock device of the present invention comprising an external clamp in an open position.

FIG. 12 depicts a side view of an exemplary lamprey lock device of the present invention comprising an external clamp in a closed position.

FIG. 13 depicts a coronal view of an exemplary lamprey lock device of the present invention comprising an external clamp in an open position.

FIG. 14 depicts a coronal view of an exemplary lamprey lock device of the present invention comprising an external clamp in a closed position.

FIG. 15 depicts a coronal view of an exemplary lamprey lock device of the present invention comprising an external lever-clamp in an open position.

FIG. 16 depicts a coronal view of an exemplary lamprey lock device of the present invention comprising an external lever-clamp in a closed position.

FIG. 17 depicts a side view of an exemplary lamprey lock device of the present invention comprising a media valve.

FIG. 18 depicts a coronal view of an exemplary lamprey lock device of the present invention comprising a media valve.

FIG. 19 depicts a coronal view of an exemplary lamprey lock device of the present invention comprising a media valve.

FIG. 20 depicts a side view of an exemplary lamprey lock device of the present invention comprising a three-way stopcock, wherein the configuration allows fluid connection between a connected catheter to the drainage reservoir.

FIG. 21 depicts a side view of an exemplary lamprey lock device of the present invention comprising a three-way stopcock, wherein the configuration allows fluid connection between a connected catheter and the flush reservoir.

FIG. 22 depicts a side view of an exemplary lamprey lock device of the present invention comprising a three-way stopcock, wherein the configuration allows fluid connection between the flush reservoir and the drainage reservoir.

FIG. 23 depicts an exemplary lamprey lock device of the present invention comprising a compressible side port valve in a close position.

FIG. 24 depicts an exemplary lamprey lock device of the present invention comprising a compressible side port valve in an open position.

FIG. 25 depicts an exemplary lamprey lock device of the present invention comprising a compressible side port valve in a close position and a flow switch in an open position.

FIG. 26 depicts an exemplary lamprey lock device of the present invention comprising a compressible side port valve in an open position and a flow switch in a close position.

FIG. 27 depicts an exemplary lamprey lock device of the present invention comprising a compressible side port valve in a close position and an external clamp in an open position.

FIG. 28 depicts an exemplary lamprey lock device of the present invention comprising a compressible side port valve in an open position and an external clamp in a close position.

FIG. 29 depicts an exemplary lamprey lock device of the present invention comprising a chamber having a side port and a compressible valve, wherein the valve is in a close position.

FIG. 30 depicts an exemplary lamprey lock device of the present invention comprising a chamber having a side port and a compressible valve, wherein the valve is in an open position.

FIG. 31 depicts an exemplary lamprey lock device of the present invention comprising a membrane within the chamber having a side port and a compressible valve, wherein the valve is in a close position.

FIG. 32 depicts an exemplary lamprey lock device of the present invention comprising a membrane within the chamber having a side port and a compressible valve, wherein the valve is in an open position.

FIG. 33 depicts an exemplary lamprey lock device of the present invention comprising a side port valve stopping flow, wherein the valve is in a close position.

FIG. 34 depicts an exemplary lamprey lock device of the present invention comprising a side port valve stopping flow, wherein the valve is in an open position.

FIG. 35 is a flowchart depicting an exemplary method of using the lamprey lock device of the present invention.

FIG. 36 depicts a drainage model experiment setup using the device of the present invention.

FIG. 37 depicts a serous model.

FIG. 38 depicts a serous 3-way model.

FIG. 39 depicts an abscess model.

FIG. 40 depicts an abscess 3-way model.

FIG. 41 depicts a cross-sectional view of an exemplary lamprey lock device of the present invention.

FIG. 42 depicts a catheter.

FIG. 43 depicts a cross-sectional view of an exemplary lamprey lock device coupled with a catheter.

FIG. 44 depicts an exemplary lamprey lock device coupled with a catheter.

FIG. 45 depicts a cross-sectional view of the exemplary lamprey lock device coupled with the catheter of FIG. 44.

FIGS. 46-48 depict exemplary lamprey lock device having a three-part assembly.

FIG. 49 depicts a cross-sectional view of the exemplary lamprey lock device of FIGS. 46-48.

FIGS. 50-52 depict an exemplary lamprey lock device having a co-molded O-ring and diaphragm.

FIGS. 53 and 54 depict the diaphragm of the exemplary lamprey lock device of FIGS. 50-52.

FIG. 55 depicts a cross-sectional view of the exemplary lamprey lock device of FIGS. 50-52.

FIGS. 56-58 depict an exemplary lamprey lock device having a co-molded O-ring and vertical diaphragm.

FIGS. 59 and 60 depict a housing of the exemplary lamprey lock device of FIGS. 56-58.

FIGS. 61 and 62 depict the vertical diaphragm of the exemplary lamprey lock device of FIGS. 56-58.

FIG. 63 depicts a cross-sectional view of the exemplary lamprey lock device of FIGS. 56-58.

FIGS. 64-66 depict an exemplary lamprey lock device having an over-molded seal and diaphragm.

FIGS. 67 and 68 depict a housing of the exemplary lamprey lock device of FIGS. 64-66.

FIGS. 69-70 depict diaphragm of the exemplary lamprey lock device of FIGS. 64-66.

FIGS. 71-73 depict an exemplary lamprey lock device having a thin diaphragm wiper.

FIG. 74 depicts a cross-sectional view of the exemplary lamprey lock device of FIGS. 71-73 coupled with a catheter.

FIGS. 75-77 depict an exemplary lamprey lock device having a ramped diaphragm.

FIG. 78 depicts a cross-sectional view of the exemplary lamprey lock device of FIGS. 75-77 coupled with a catheter.

FIGS. 80, and 83-86 depict an exemplary lamprey lock device having an overmolded seal and a diaphragm with a wiper having a reduced undercut.

FIG. 81 depicts an integration of a stepped portion in a seal of a lamprey lock device.

FIG. 82 depicts a thread stop on the housing of the lamprey lock device of FIG. 81.

FIGS. 87 and 88 depict compression between the exemplary lamprey lock device of FIGS. 81 and 82 and a catheter.

FIGS. 89-91 depict an exemplary lamprey lock device having an overmolded seal and a diaphragm wiper.

FIG. 92 depicts an exemplary lamprey lock device having an overmolded seal and a diaphragm wiper with an alternative grip.

FIGS. 93 and 95 depict a top portion and a bottom portion of a housing of an exemplary lamprey lock device.

FIG. 94 depicts an overmolded diaphragm on the top portion of the housing.

FIG. 96 depicts a seal on the bottom portion of the housing.

FIG. 97 depicts a cross-sectional view of an assembled exemplary lamprey lock device having an overmolded diaphragm and a seal.

FIG. 98 depicts a cross-sectional view of an assembled exemplary lamprey lock device having an overmolded seal and a grip-diaphragm wiper.

FIGS. 99 and 100 are tables describing mechanical properties of the exemplary lamprey lock device.

FIGS. 101 and 102 are tables illustrating results of separation of the exemplary lamprey lock device from an axial load.

FIGS. 103-105 are tables illustrating results of separation of the exemplary lamprey lock device from unscrewing.

FIGS. 106 and 107 are tables illustrating results of the exemplary lamprey lock device from resistance to overriding.

FIGS. 108-111 are tables illustrating tubing detachment force applied to the exemplary lamprey lock device.

FIGS. 112 and 113 are tables illustrating positive pressure liquid leakage testing.

FIGS. 114-116 depict an initial pressure test setup for the exemplary lamprey lock device connectors and a table illustrating results of fluid leakage performance with a positive pressure liquid leakage test.

FIGS. 117 and 118 depict a secondary pressure test setup for the exemplary lamprey lock device. FIGS. 119-124 are tables illustrating results of liquid leakage pressure testing.

FIGS. 125-127 depict various views of a luer lock cap.

FIG. 128 depicts a pressure test setup for the luer lock cap.

FIGS. 129-132 are tables illustrating results of the luer lock cap pressure testing.

FIG. 133 depicts an isometric view of an exemplary lamprey lock device having an anti-rotation feature and a grip feature.

FIG. 134 depicts a side view of the exemplary lamprey lock device.

FIG. 135 depicts another side view of the exemplary lamprey lock device and the anti-rotation feature.

FIG. 136 depicts a top view of the exemplary lamprey lock device.

FIG. 137 depicts a bottom view of the exemplary lamprey lock device.

FIGS. 138 and 139 depict cross-sectional views of the exemplary lamprey lock device.

FIG. 140 depicts a catheter coupled to an exemplary lamprey lock device.

FIGS. 141 and 142 depict cross-sectional views of the catheter coupled to the exemplary lamprey lock device.

FIGS. 144 and 145 depict cross-sectional views of Luer Stopcocks respectively coupled to an exemplary lamprey lock device.

FIGS. 146a and 146b depicts an exemplary lamprey lock device. FIGS. 147a and 147b depicts another exemplary lamprey lock device. FIGS. 148-151 depict unscrewing resistance forces applied to the exemplary lamprey lock device.

FIGS. 152-155 depicts an exemplary lamprey lock device.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of adaptors. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Lamprey Lock

The present invention provides a lamprey lock device configured to provide a fluid transfer between two devices or objects. In one embodiment, lamprey lock device of the present invention improves fluid transfer by maximizing the inner diameter of connections between two objects including but not limited to a catheter, tubing, veress needles, trocars, syringes, or gas/fluid delivery systems. In one embodiment, lamprey device of the present invention is fully compatible with two objects utilizing a female luer lock distal end. In one embodiment, the lamprey device of the present invention provides a connection between a catheter at its proximal end to tubing including but not limited to a drainage reservoir tubing. In one embodiment, the lamprey device of the present invention allows health care institutions to continue using current catheter designs with the added benefit of increasing minimal inner diameter at connections with the lamprey lock device. In one embodiment, while maintaining compatibility, cross-sectional area of connections with the lamprey lock device can be maximized to 13 mm² compared to 3 mm² with traditional luer lock connections.

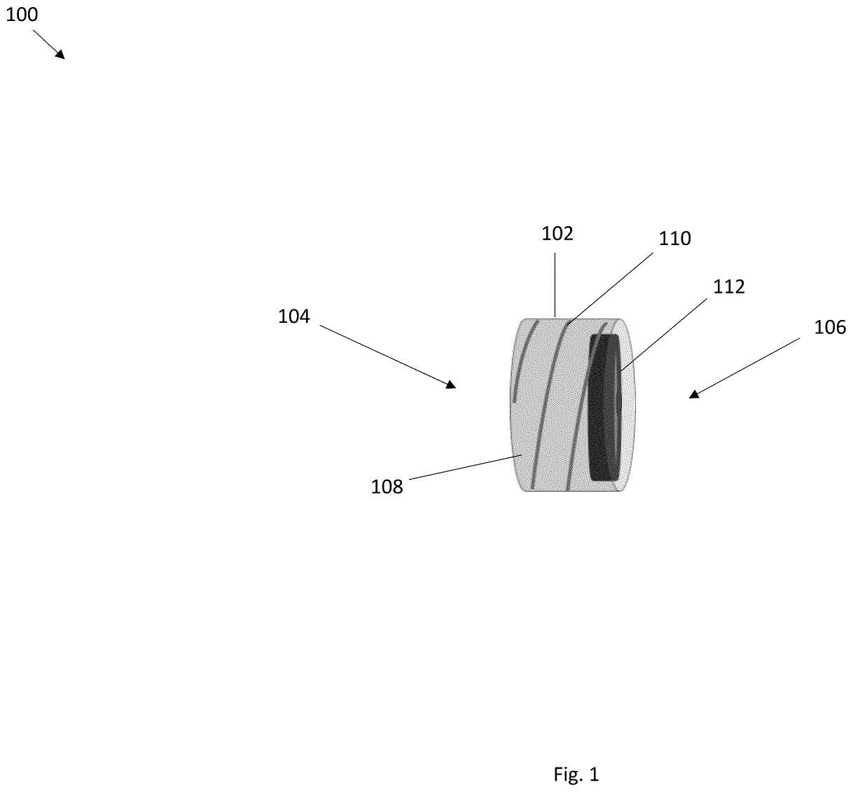

Referring now to FIG. 1, an exemplary lamprey lock device 100 of the present invention is shown. Lamprey lock device 100 comprises a luer taper adapter 102 having a proximal end 104, a distal end 106 and forming a lumen 108 therebetween. The inner surface forming lumen 108 may be smooth, barbed, tapered, threaded, or have any other surface topography to assist securing a conduit to it. In one embodiment, the inner surface of lumen 108 may comprise a plurality of threads 110 on all or a portion of the interior wall within lumen 108 of luer taper adapter 102. In one embodiment, plurality of threads 110 may be a left-handed helical thread. In one embodiment, plurality of threads 110 may be a right-handed helical thread. As contemplated herein, lumen 108 may have an internal, cross-sectional diameter between 1-8 mm. In some embodiments, lumen 108 may have a cross-sectional diameter between 6-8 mm. In some embodiments, the cross sectional diameter of lumen 108 is equal or greater than 1 mm, equal or greater than 2 mm, equal or greater than 3 mm, equal or greater than 4 mm, equal or greater than 5 mm, equal or greater than 6 mm, equal or greater than 7 mm, or equal or greater than 8 mm. In one embodiment, lumen 108 and plurality of threads 110 may be sized to comply with the widely used 6% luer taper used for intravascular and hypodermic applications (international Organization of Standard (“ISO”) standards 80369-7). In one embodiment, lumen 108 may comprise any number of thread configurations available to provide and interlock between medical devices. In one embodiment, plurality of threads 110 have a spiral rotation of at least 0.5. It should be appreciated that there is no limitation to the size, profile, and pitch of threads 110 of luer taper 102.

In one embodiment, lumen 108 may have a length ranging between 1-12 mm. In some embodiments, lumen 108 may have a length between 8 mm and 10 mm. In some embodiments, the length of 108 is equal or greater than 1 mm, equal or greater than 2 mm, equal or greater than 3 mm, equal or greater than 4 mm, equal or greater than 5 mm, equal or greater than 6 mm, equal or greater than 7 mm, equal or greater than 8 mm, equal or greater than 9 mm, or equal or greater than 10 mm.

Luer taper adapter 102 may further include an O-ring 112, positioned within lumen 108, configured to create a watertight seal with the proximal end of the inserted object including but not limited to a catheter. In one embodiment, O-ring 112 may be positioned at or near distal end 106 of lumen 108. In one embodiment, O-ring 112 may comprise rubber, silicone, PVC, or any other material operative to provide a seal to the surface of the mating connector to prevent fluids or gasses from leaking around O-ring 112 or passing through O-ring 112 itself. In one embodiment, O-ring 112 may have a maximum outer diameter of 8 mm. In one embodiment, O-ring 112 may have a maximum inner diameter of 4 mm.

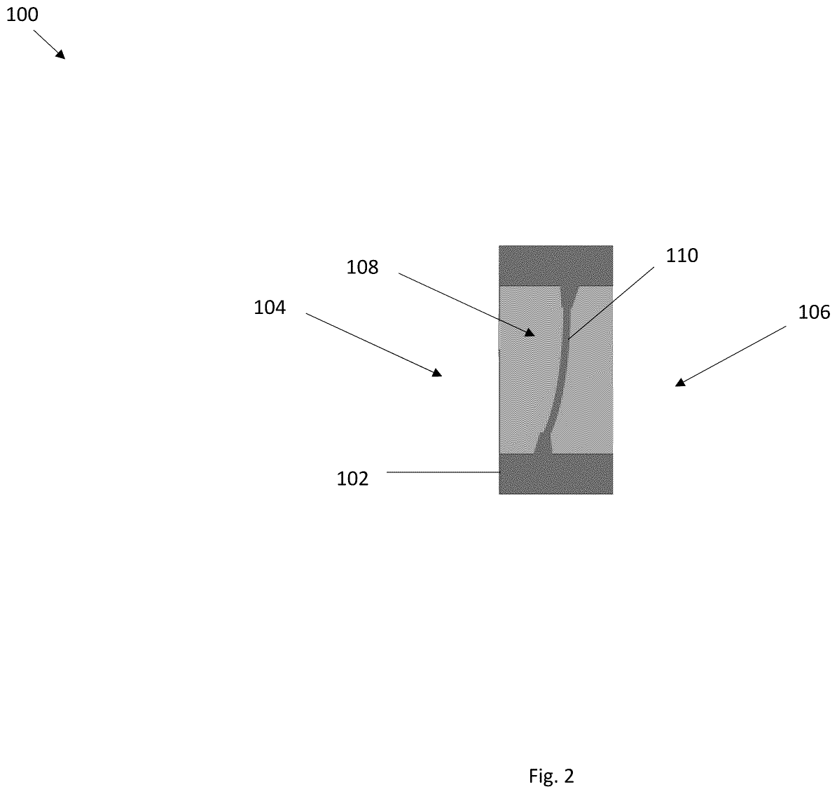

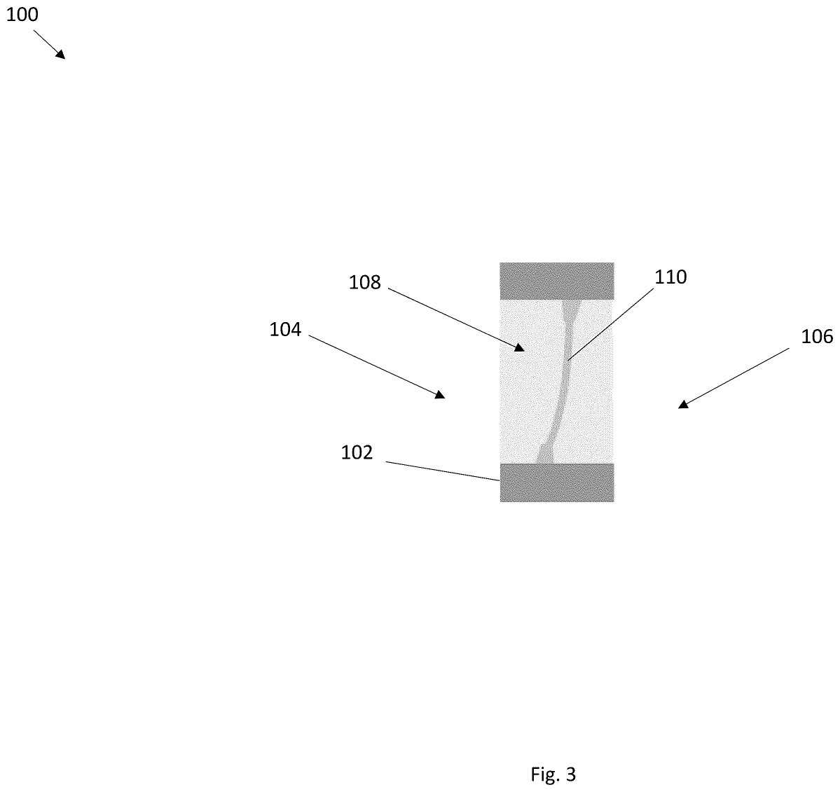

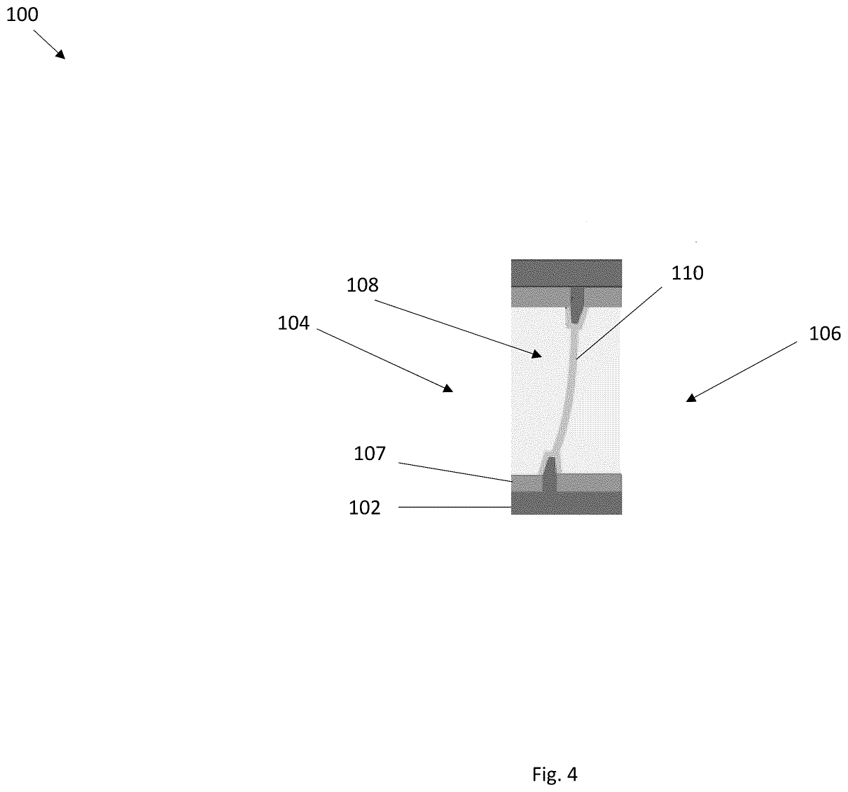

Luer taper adapter 102 may be made from any suitable material including but not limited to metal, plastic, an elastomeric material, or combinations thereof (FIG. 2, FIG. 3, and FIG. 4). In one embodiment, luer taper adapter 102 may be made from a rigid material. In one embodiment, luer taper adapter 102 may be made from an elastomeric material. In one embodiment, the inner surface of luer taper adapter 102 may be covered with a covering or coating 107. In one embodiment, coating or covering 107 may be elastomeric. In one embodiment, covering or coating 107 may be a rigid plastic. Covering or coating 107 is configured to cover the inner surface of luer taper adapter 102 within lumen 108 to create a watertight seal. In one embodiment, plastic materials for any components of device 100 may include but are not limited to polycarbonate, polyethylene, polypropylene, polyesters, and co-polyesters. In one embodiment, elastomeric material for any components of device 100 may include but are not limited to silicone, silicone rubber, or polyisoprene.

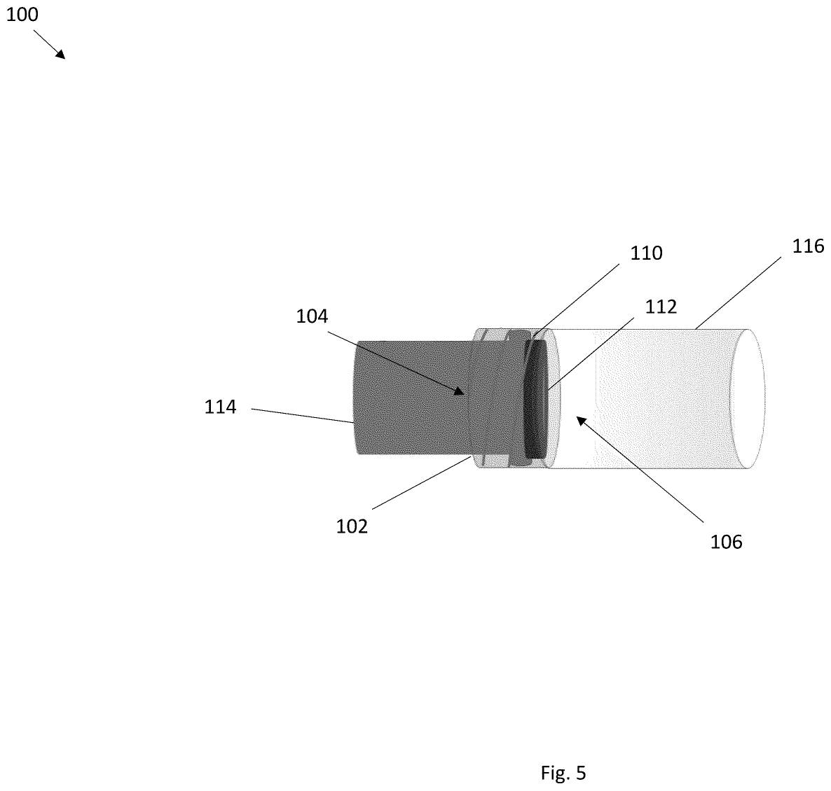

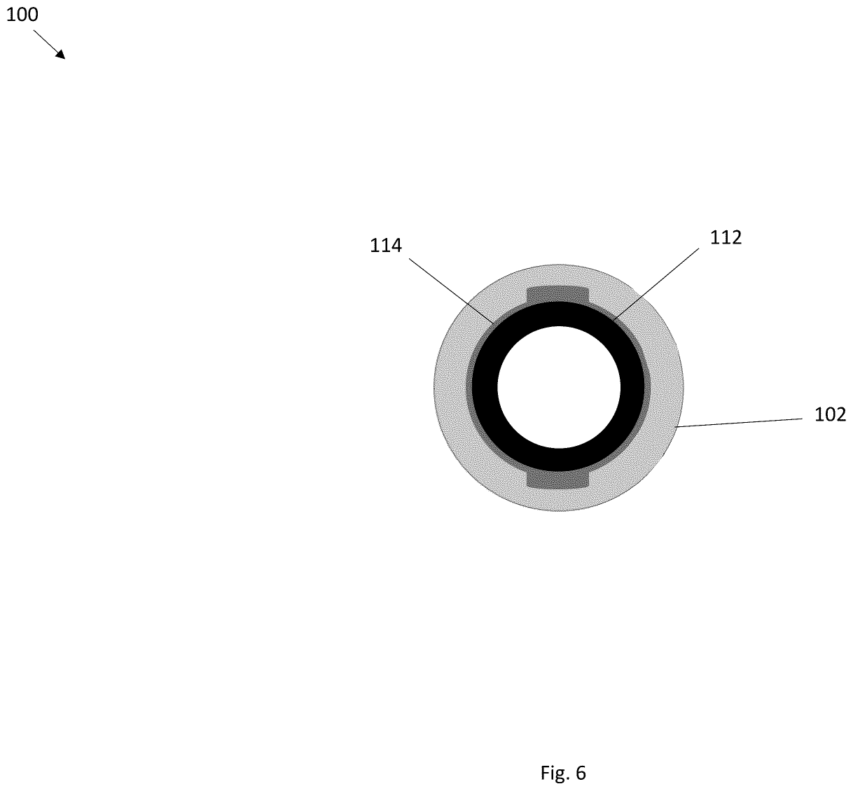

Referring now to FIG. 5 and FIG. 6, an exemplary catheter 114 connection with luer taper adapter 102 is shown. In one embodiment, a catheter 114 may be inserted into lumen 108 at proximal end 104, engaging plurality of threads 110 with clockwise rotation to create a watertight seal with O-ring 112 at distal end 106. Catheter 114 may be disconnected from luer taper adapter 102 with a counterclockwise rotation. In other embodiments, any other medical device may be inserted at proximal end 104 with clockwise rotation. In one embodiment, catheter 114 may be any catheter including but not limited to drainage catheter, vascular access, urinary and biliary catheter. It should be appreciated that any similarly tube-shaped and/or tapered device that permits flow therethrough (flexible, rigid expandable and/or extendable) may be suitable for insertion into proximal end 104. In one exemplary embodiment, a tubing 116 may be inserted at distal end 106. Tubing 116 may be attached to the outer surface of lumen 108 at distal end 106.

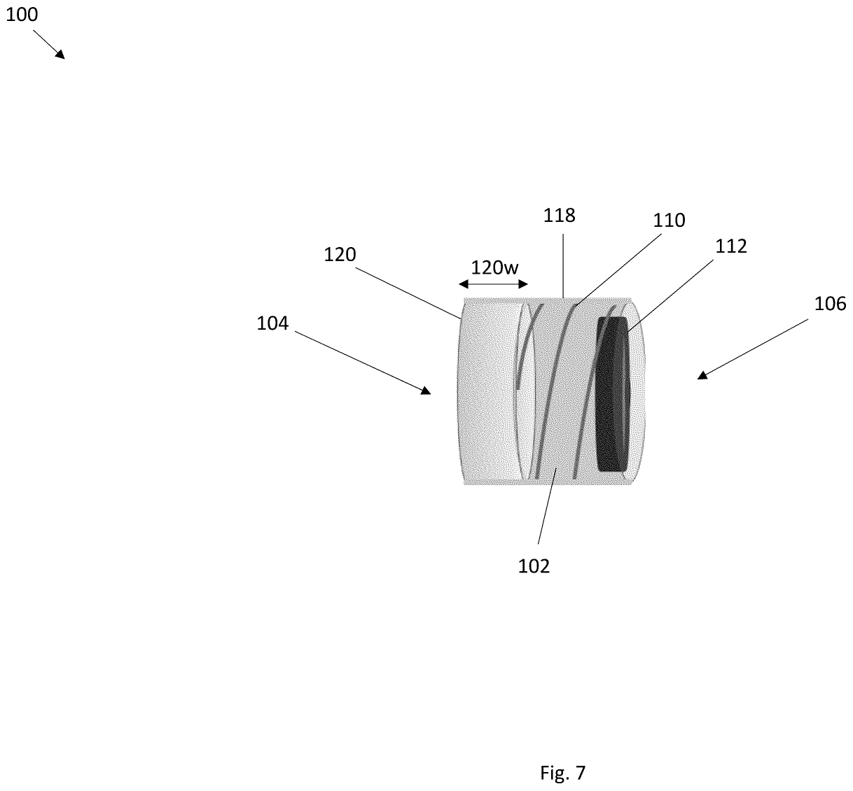

In one embodiment, lamprey lock device 100 may further comprise a covering or housing 118 (FIG. 7), configured to secure components of device 100 together. In some embodiments, covering or housing 118 may be generally tubular or cylindrical in shape. However, it should be appreciated that there are no limitations to the shape of covering 118, such that the internal components of device 100 can be positioned securely therein. In one embodiment, covering 118 may be made from materials including but not limited to metal, plastic, an elastomeric material, or combinations thereof.

In one embodiment, lamprey lock device 100 may further comprise a circumferential elastomeric seal 120 positioned at or near proximal end 104, configured to create an additional watertight seal around the inserted medical device including but not limited to a catheter. In one embodiment, circumferential elastomeric seal 120 is configured to create an additional watertight seal in the event the user has not fully tightened the medical device within the luer taper adapter portion 102. In one embodiment, elastomeric seal 120 may be made from materials including but not limited to rubber, silicone and etc. In one embodiment, the elastomeric seal may be composed of an open cell matrix made from material including but not limited to polyurethane, polyethylene and etc. In one embodiment, circumferential elastomeric seal 120 has an inner diameter ranging between 4-8 mm. In some embodiments, the circumferential elastomeric seal 120 has an inner diameter ranging from 5-7 mm. In some embodiments, circumferential elastomeric seal 120 inner diameter is equal or greater than 4 mm, equal or greater than 5 mm, equal or greater than 6 mm, equal or greater than 7 mm, or equal or greater than 8 mm.

In one embodiment, circumferential elastomeric seal 120 has a thickness ranging between 1-6 mm. In some embodiments, the circumferential elastomeric seal 120 has a thickness ranging from 2-4 mm. In some embodiments, circumferential elastomeric seal 120 has a thickness equal or greater than 1 mm, equal or greater than 2 mm, equal or greater than 3 mm, equal or greater than 4 mm, equal or greater than 5 mm, or equal or greater than 6 mm.

In one embodiment, circumferential elastomeric seal 120 has a width 120w ranging between 1-6 mm. In some embodiments, the circumferential elastomeric seal 120 has a width ranging from 1-3 mm. In some embodiments, circumferential elastomeric seal 120 has a width equal or greater than 1 mm, equal or greater than 2 mm, equal or greater than 3 mm, equal or greater than 4 mm, equal or greater than 5 mm, or equal or greater than 6 mm.

In one embodiment, covering 118 is positioned around luer taper adapter 102 and circumferential elastomeric seal 120 to secure them together.

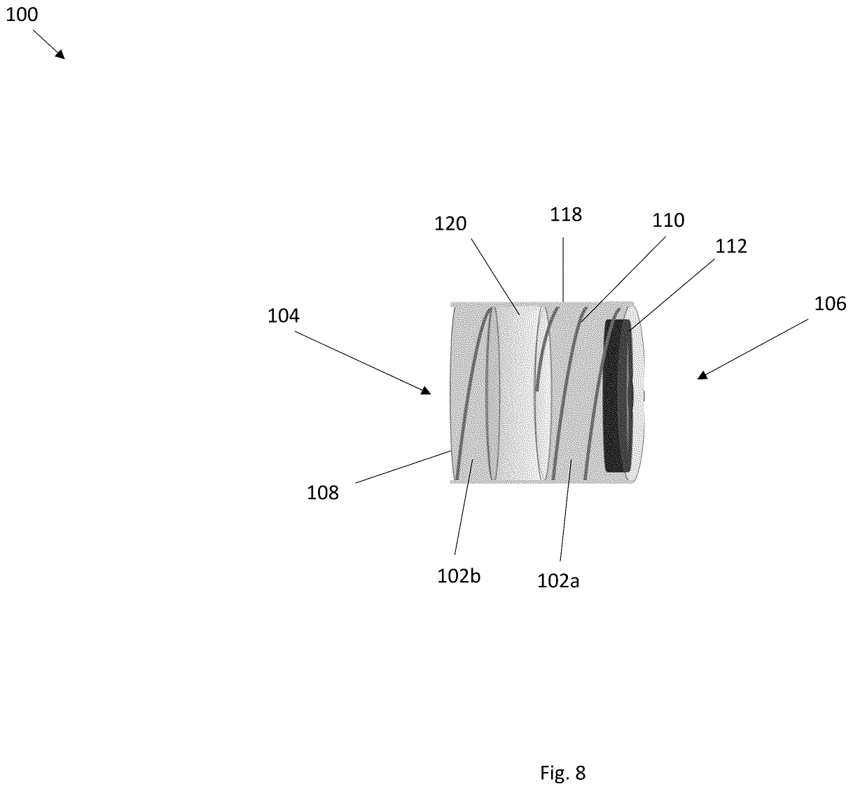

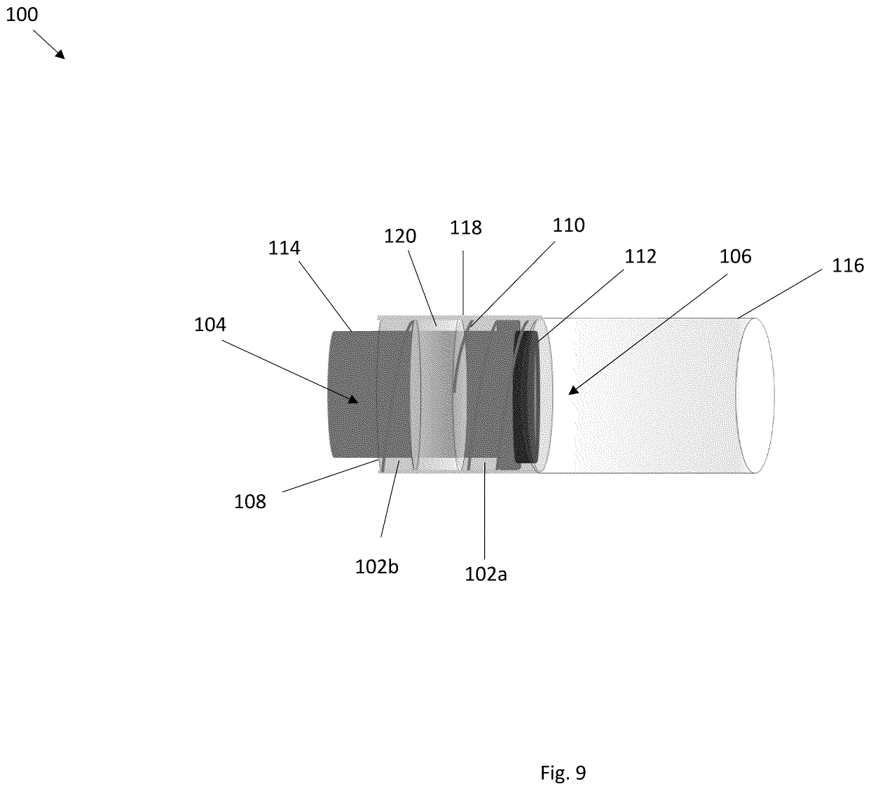

Referring now to FIG. 8, in one embodiment, a second luer taper adapter 102b may be positioned proximal to circumferential elastomeric seal 120, and held together with covering 118. In one embodiment, rotational motion allows for catheter 114 to pass through circumferential elastomeric seal 120 more easily. In one embodiment, when catheter 114 is advanced through the luer taper adapters 102a and 102b and circumferential elastomeric seal 120, watertight seals are created at the side of catheter 114 and at distal end 106 to prevent the leakage of fluid (FIG. 9). It should be appreciated that device 100 may include one or a plurality of luer taper adapters, and zero, one or a plurality of elastomeric seals. It should further be appreciated that the configuration of luer taper portions and elastomeric seal portions can be in any combination. For example, device 100 may include one or more luer tapers, and/or one or more elastomeric seals. For example, In one embodiment, device 100 may include, from proximal end to distal end, a single luer taper adapter and an O-ring. In another embodiment, device 100 may include, from proximal end to distal end, an elastomeric seal, a luer taper adapter and an O-ring. In another embodiment, device 100 may include, from proximal end to distal end, a first luer taper adapter, elastomeric seal, a second luer taper adapter, and an O-ring. In another embodiment, device 100 may include, from proximal end to distal end, a first luer taper adapter, a second luer taper adapter, and an O-ring, where the first and second luer taper adapters have a space between them. In another embodiment, device 100 may include, from proximal end to distal end, an elastomeric seal, a first luer taper adapter, a second luer taper adapter, and an O-ring, where the first and second luer taper adapters have a space between them. It should further be appreciated that the O-ring, luer taper adapters and elastomeric seals when adjacent to each other may be in direct contact or spaced apart, in any combination.

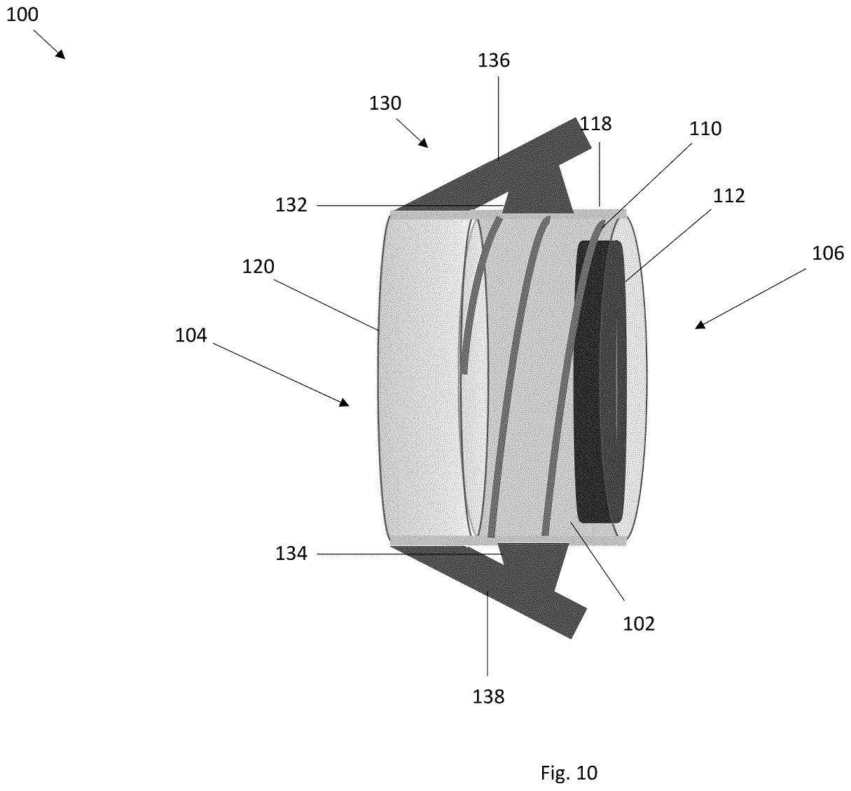

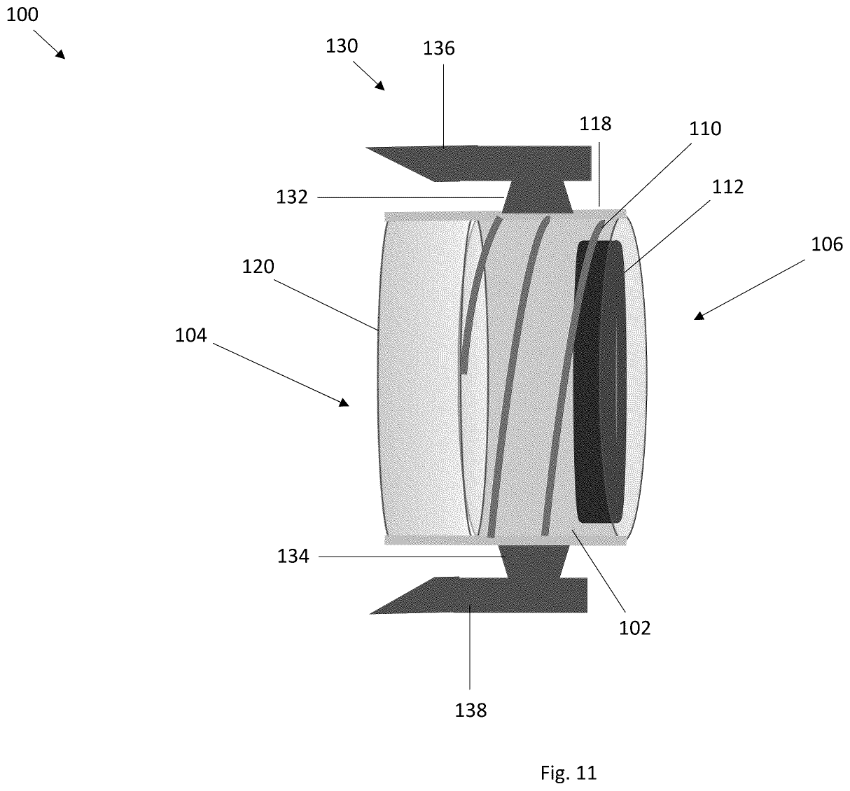

In one embodiment, lamprey lock device 100 may further comprise an external clamp 130 positioned around covering 118 on top of elastomeric seal 120, configured to allow insertion and withdrawal of catheter 114 or any other medical device from device 100. In one embodiment, external clamp 130 may be fastened around elastomeric seal 120 by any methods known to one skilled in the art. In one embodiment, external clamp 130 comprises a first attachment point 132, a second attachment point 134, a first articulating arm 136 having a proximal end 135, a distal end 137 and a length therebetween, and a second articulating arm 138 having a proximal end 139, a distal end 141 and a length therebetween. First attachment point 132 and second attachment point 134 are attached to the outer surface of covering 118 and are hingedly connected to first articulating arm 136 and second articulating arm 138. In one embodiment, first attachment point 132 and second attachment point 134 may be connected to first articulating arm 136 and second articulating arm 138 at any point on their length, such that pushing one end of articulating arms 136 and 138, causes the other end to move in the opposite direction and thereby release from pushing circumferential elastomeric seal 120 down and create a seal (FIG. 10 and FIG. 11).

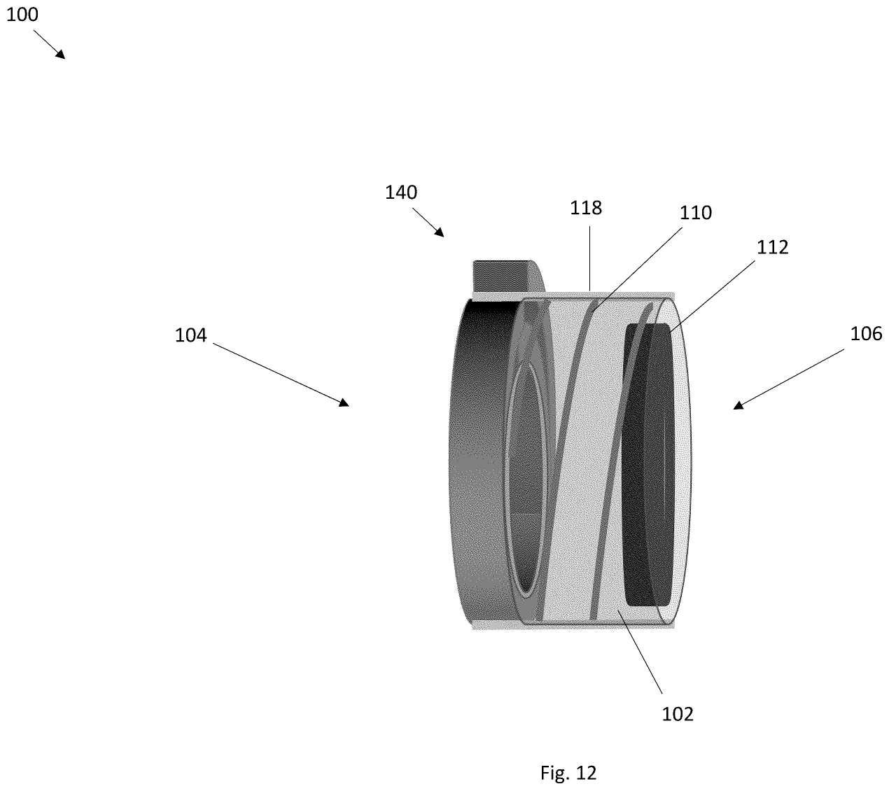

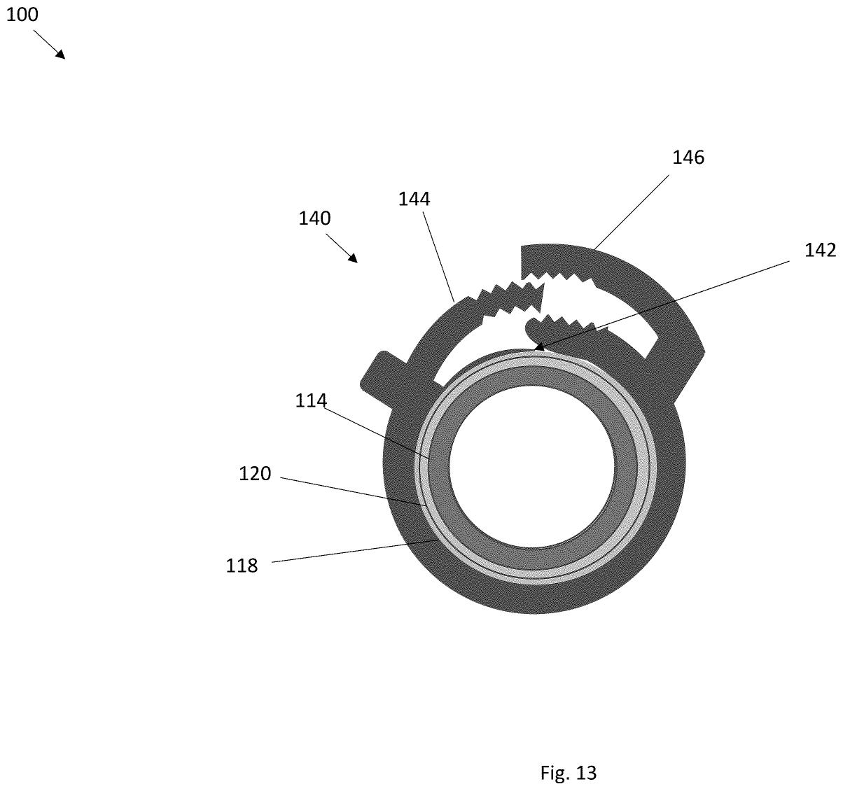

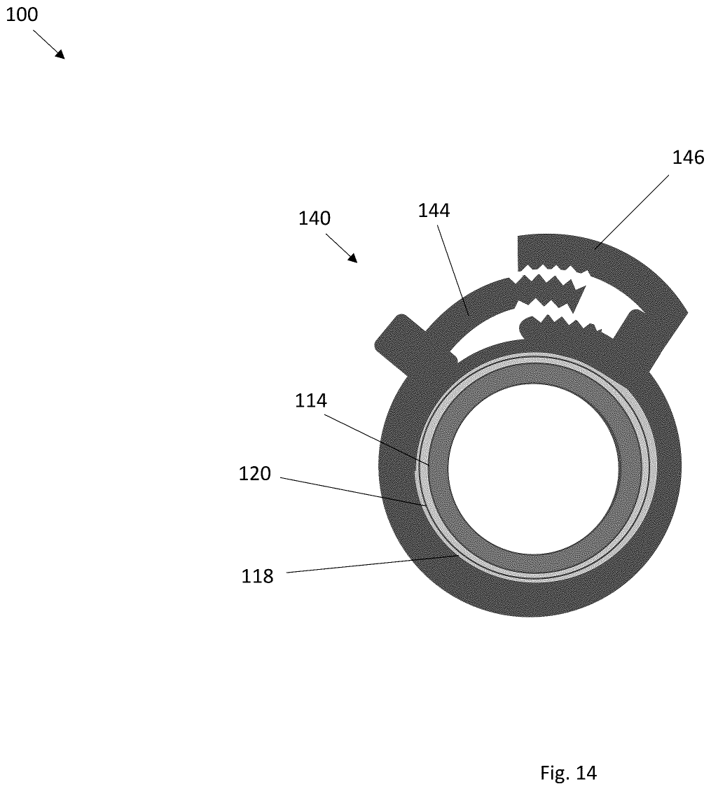

Referring now to FIG. 12 through FIG. 14, another exemplary external clamp 140 is shown. External clamp 140 may comprise an opening 142 for inserting circumferential elastomeric seal 120 therein, a first end 144 and a second end 146. First end 144 and second end 146 may comprise one or more tightening elements to allow fasten and tighten the clamp by applying a radially inward clamping force on the circumferential elastomeric seal 120 inserted in opening 142. In one embodiment, first end 144 and second end 146 may be interlocking arms.

In one embodiment, first end 144 may comprise a single ridge arm and second end 146 may comprise a double ridge arm structure, such that the single ridge can be secured and fastened between the double ridge and lead to locking of clamp 140 around circumferential elastomeric seal 120.

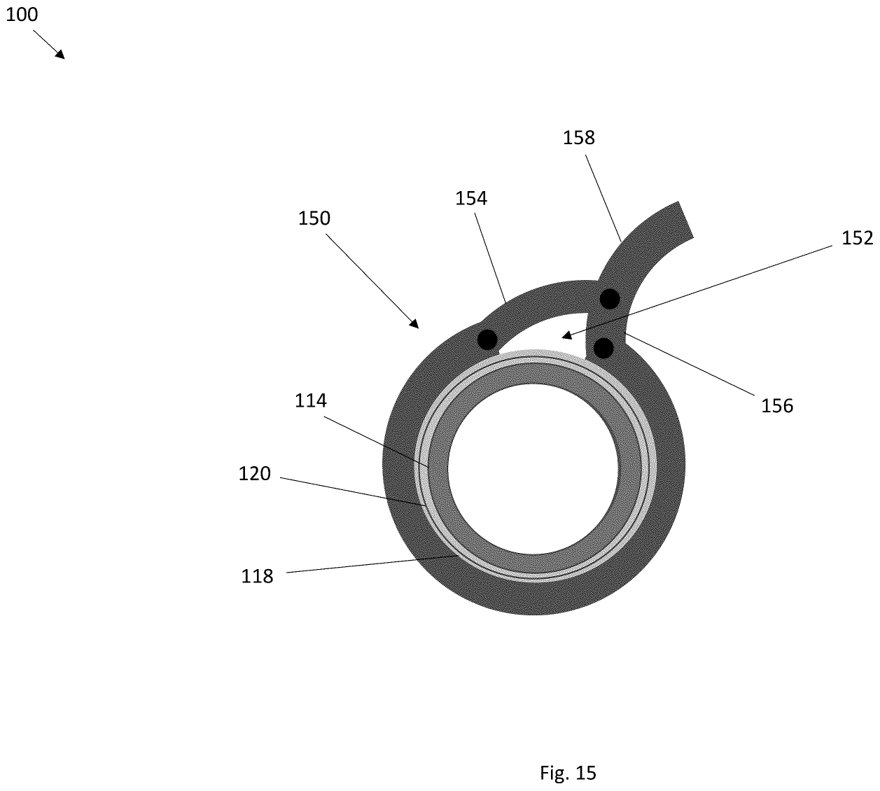

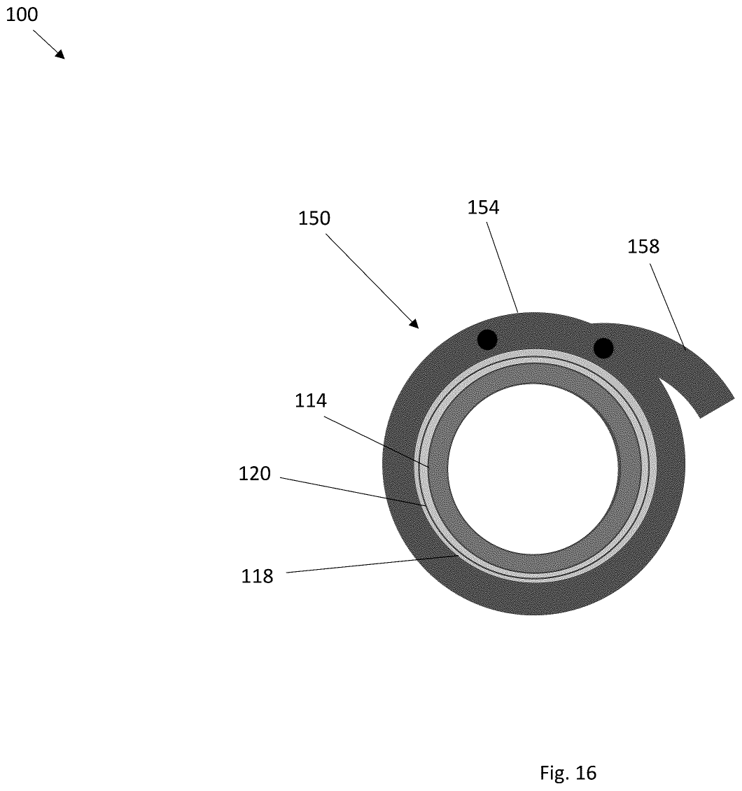

Referring now to FIG. 15 and FIG. 16, another exemplary external clamp 150 is shown. External clamp 150 may be a lever-type clamp. External clamp 150 may comprise an opening 152 for inserting circumferential elastomeric seal 120 therein, a first end 154 and a second end 156. First end 154 and second end 156 may be hingedly connected to each other and a lever 158, such that upon manipulation of the lever in one direction first end 154 and second end 156 can be drawn tight around circumferential elastomeric seal 120.

In one embodiment, external clamp 130 may be made with any suitable material known to one skilled in the art including but not limited to a flexible metal sheet, a flexible plastic, etc.

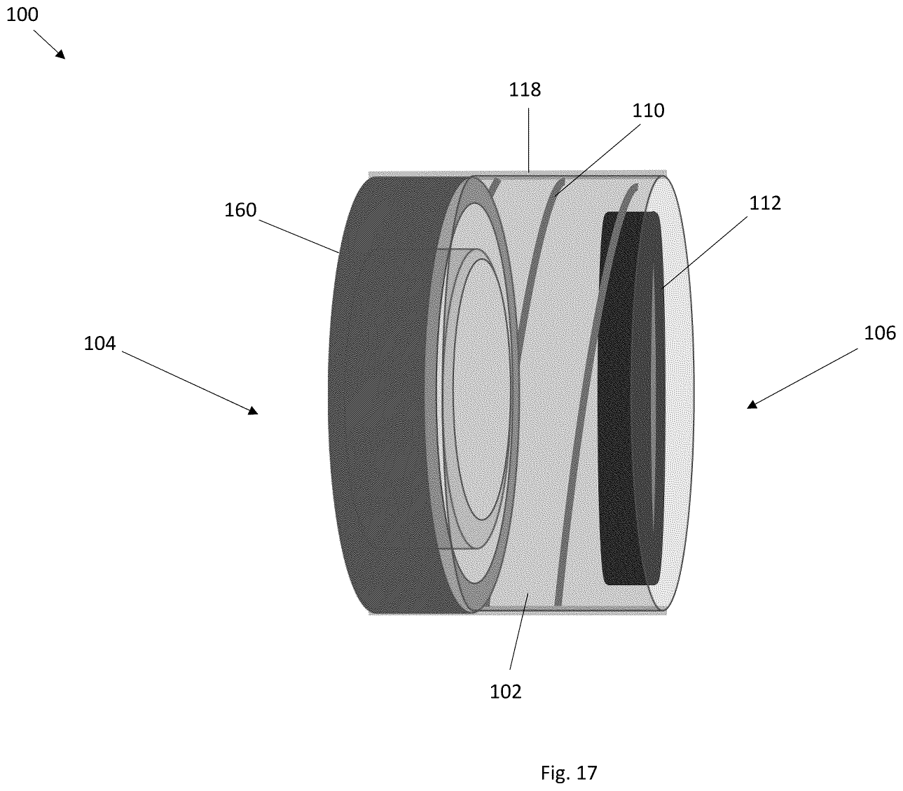

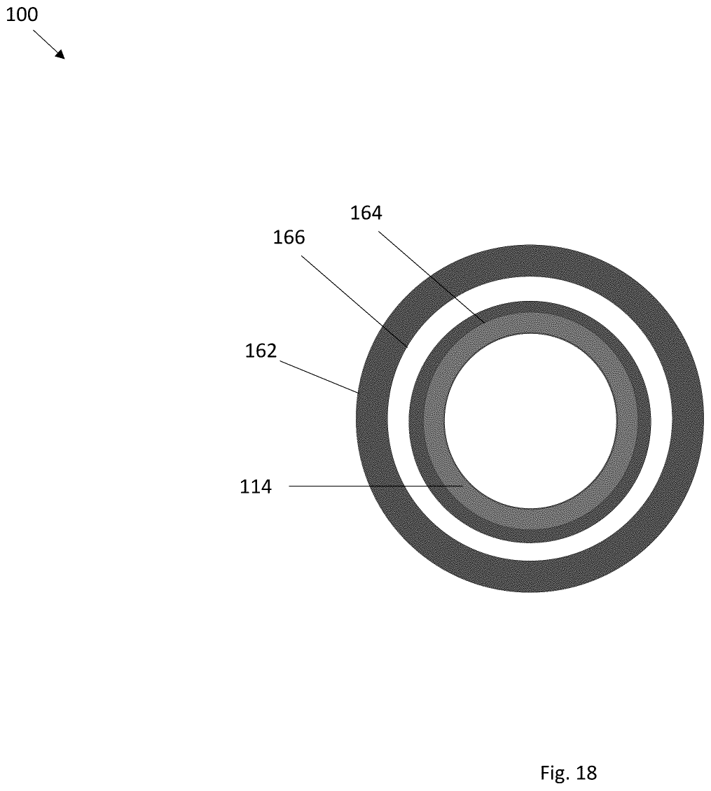

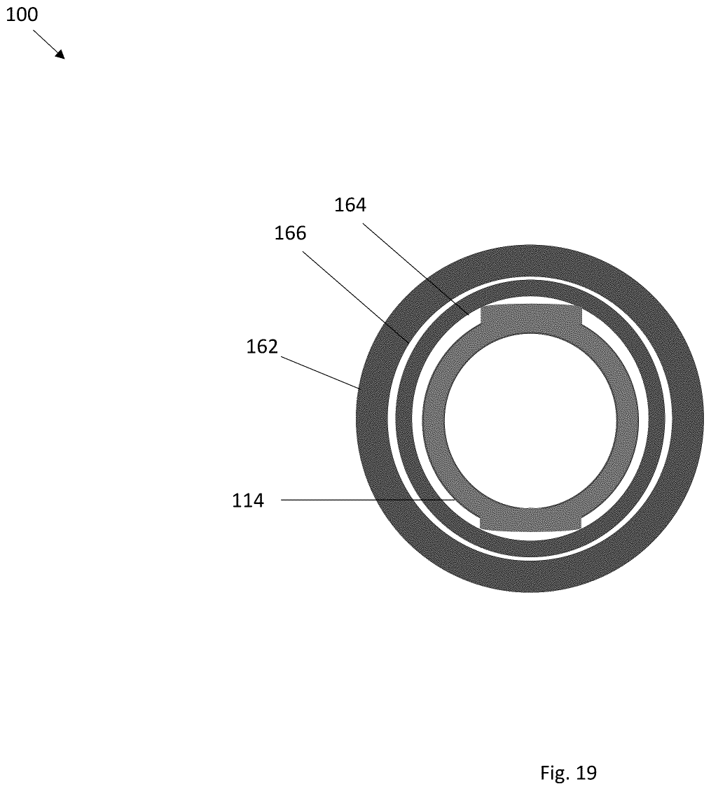

Referring now to FIG. 17, a side view of an exemplary lamprey lock device 100 of the present invention is shown. In one embodiment, lamprey lock device 100 may further comprise media valve 160 positioned at proximal end 104, configured to create an additional watertight seal around the medical device including but not limited to a catheter. In one embodiment, media valve 160 is configured to create an additional watertight seal in the event the user has not fully tightened the medical device attached to proximal end 104. In one embodiment, media valve 160 may be made from materials including but not limited to rubber, silicone and etc. In one embodiment, media valve 160 has an inner diameter ranging between 5-8 mm. In one embodiment, covering 118 is positioned around luer taper adapter 102 and media valve 160 to secure them together. Referring now to FIG. 18 and FIG. 19, a top view of an exemplary catheter 114 connection with luer taper adapter 102 comprising media valve 160 is shown. Media valve 160 comprises an outer surface 162, an inner surface 164 and a media chamber 166 positioned in between. Media chamber 166 may be inflated to seal create a watertight seal around catheter 114. In one embodiment, media chamber 166 may be inflated with any suitable media known to one skilled in the art including but not limited to water, saline, air, etc. In one embodiment, media chamber 166 may be filled with media by any suitable method known to one skilled in the art including but not limited to tubing, etc. Inner surface 164 may be compressed to allow insertion of catheter 114 into luer taper adapter 102. In one embodiment, media chamber 166 may be inflated and deflated to allow insertion and removal of catheter 114 from luer taper adapter 102.

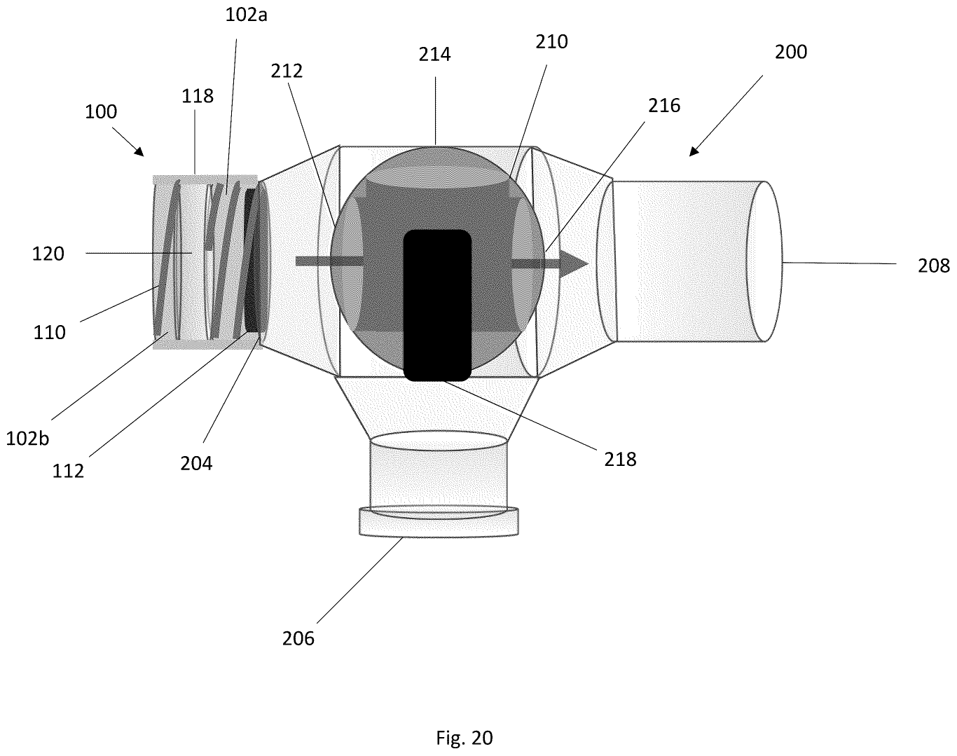

Referring now to FIG. 20, in one embodiment, lamprey lock device 100 may be connected to a three-way stopcock 200. Three-way stopcock 200 comprises a tubular shape comprising a first tube section, a second tube section and a third tube section, each tube having a lumen defined by a circumference, and each tube section fluidly joined to each other at a central connector. The first tube section terminates in an open end 204 opposite to the central connector, the second tube section terminates in an open end 206 opposite to the central connector, and the third tube section terminates in an open end 208 opposite to the central connector. Stopcock 200 enables fluid communication between drainage reservoir and flush reservoir with single lumen catheters by selectively switching fluid access between a catheter with a drainage reservoir and a flush reservoir. The single lumen catheter is thereby configured to act as both a flush lumen and a drainage lumen. In one embodiment, the catheter is any standard drainage catheter known in the art.

Open end 204 is fluidly connected to distal end 106 of lamprey lock device 100 as described elsewhere herein. In one embodiment, open end 204 has the same diameter as lumen 108 of lamprey lock device 100.

In one embodiment, open end 206 is fluidly connected to a flush syringe. In one embodiment, open end 206 is fluidly connected to a flush pump. In one embodiment, open end 206 comprises a female luer adapter side port to allow insertion and withdrawal of fluids. In one embodiment, open end 208 is fluidly connected to a drainage reservoir. In one embodiment, the fluid connections can be any suitable mechanism known in the art configured to fluidly join tubes end-to-end, including but not limited to luer locks, tube fittings, threaded connectors, barbed connectors, and the like.

In one embodiment, direction of fluid flow in stopcock 200 can be adjusted manually. In one embodiment, direction of fluid flow in stopcock 200 can be adjusted electronically.

In one embodiment, stopcock 200 further comprises a central component 210 positioned within the central connector. Central component 210 comprises a circumference that is slightly less than the circumference of stopcock 200. As such, central component 210 may be inserted within stopcock 200 with minimal clearance. When inserted within stopcock 200, the minimal tolerance between the outer surface of central component 210 and the inner surface of stopcock 200 prevents fluid from leaking to drainage reservoir or flush reservoir. Central component 210 comprises a first open side 212, a second open side 214, a third open side 216 and a closed side 218 and is provided to allow directional adjustment to the fluid pathway. In one exemplary embodiment shown in FIG. 20, first open side 212 is in alignment with open end 204, wherein second open side 214 is faced to the wall of stopcock 200, third opening 216 is in alignment with open end 208 and closed side 218 is in alignment with open end 206. In this exemplary configuration, central component 210 allows fluid connection between a connected catheter to open end 204 and a drainage reservoir and blocks fluid communication from the flush reservoir to the catheter.

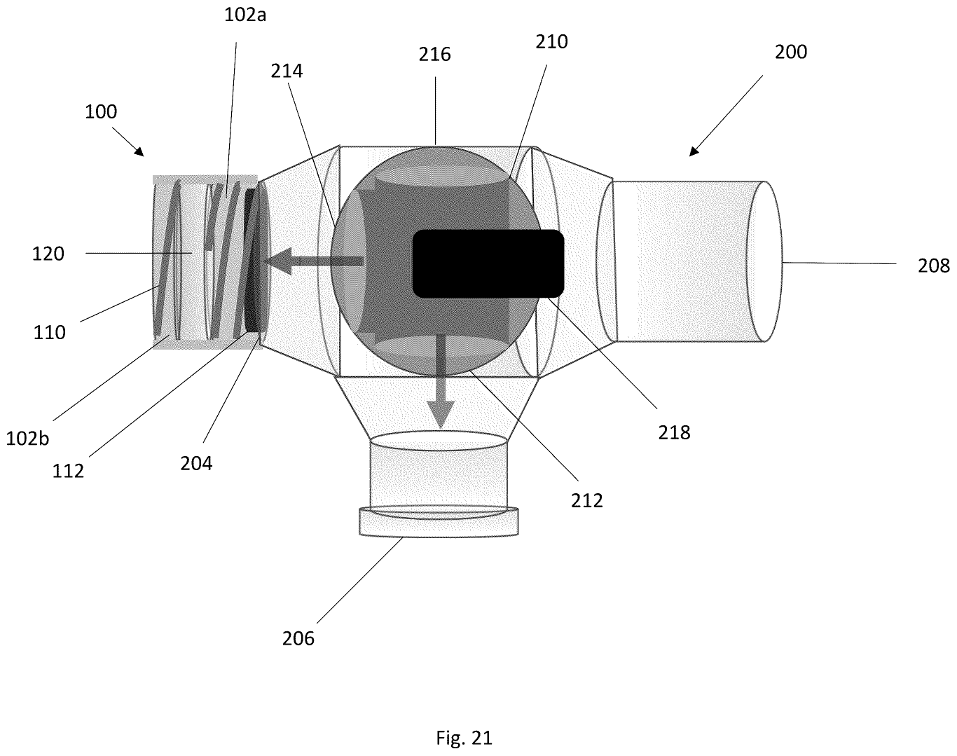

In operation, stopcock 200 comprises a drain mode and a flush mode. In the drain mode (depicted in FIG. 20), central component 210 produces a patent channel from the catheter through first open side 212 and third open side 216 into a drainage reservoir, while closed side 218 blocks fluid communication to a flush reservoir. In flush mode (as depicted in FIG. 21), central component 210 produces a patent channel from the flush reservoir through first open side 212 and second open side 214 into the catheter connected to lamprey lock device 100, while closed side 218 blocks fluid communication to the drainage reservoir. Central component 210 can be controlled manually or electronically to switch stopcock 200 between the drain mode and the flush mode.

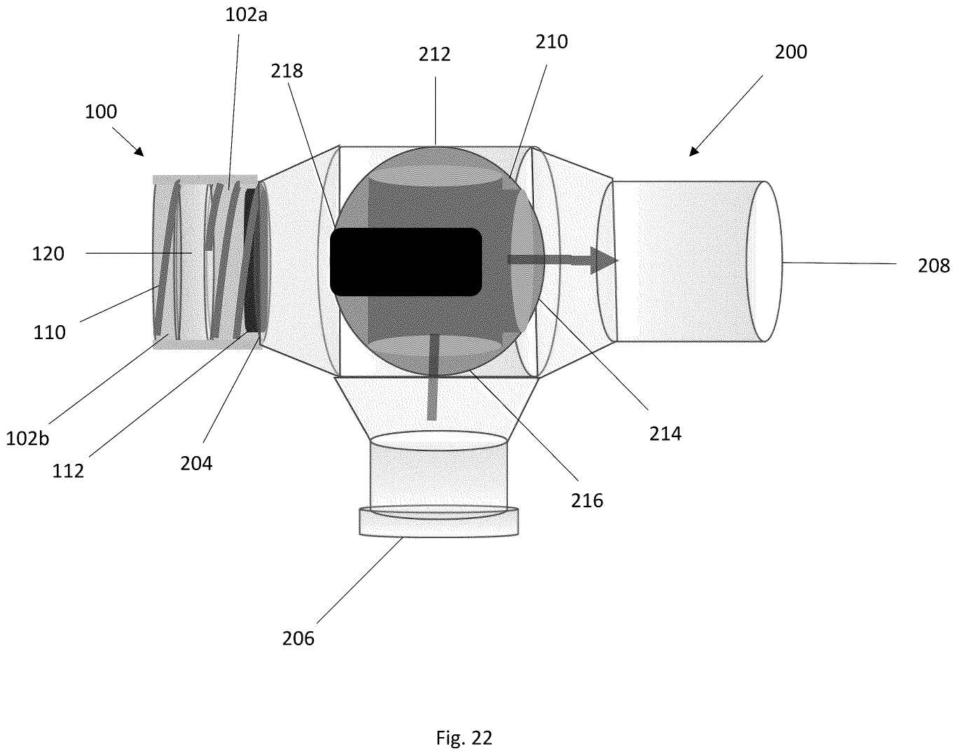

Stopcock 200 may also produce a patent channel from the flush reservoir to drainage reservoir through second open side 214 to third open side 216 as shown in FIG. 22.

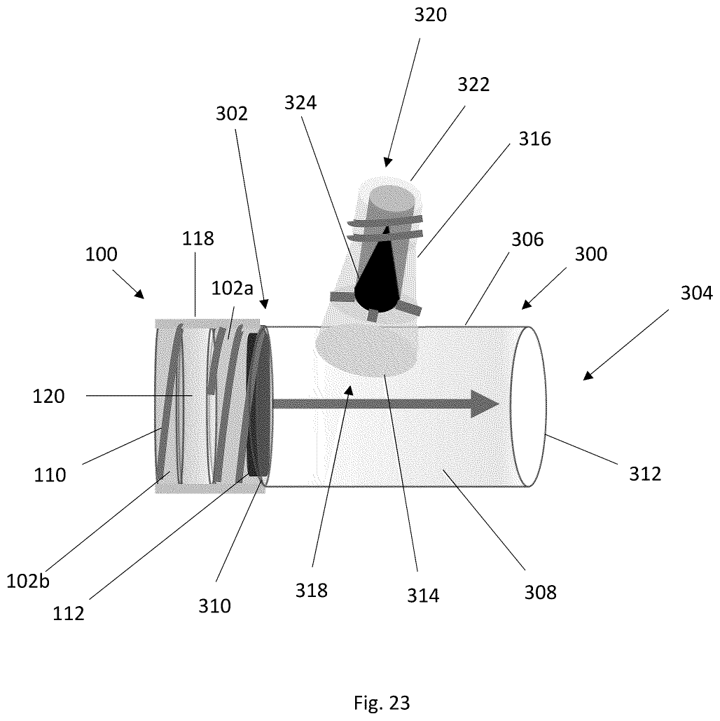

Referring now to FIG. 23, in one embodiment, lamprey lock device 100 may be connected to a tubing 300 at distal end 106, wherein tubing 300 comprises a proximal end 302, a distal end 304 and a body 306 therebetween. Body 306 has an elongated cylindrical shape comprising a lumen 308 with a diameter ranging between 2-5 mm. In one embodiment, body 306 is approximately 5-16 mm in length, however any suitable length may be used. Body 306 has a first opening 310 at proximal end 302, a second opening 312 at distal end 304 and a third opening 314. In one embodiment, third opening 314 is located between first opening 310 and second opening 312 at any suitable distance away from first opening 310 and second opening 312. In one embodiment, the distance between first opening 310 and third opening 314 is more than the distance between the second opening 312 and the third opening 314. In one embodiment, the distance between first opening 310 and third opening 314 is less than the distance between the second opening 312 and the third opening 314. In one embodiment, first opening 310 and second opening 312 have an inner diameter ranging between 4-5 mm. In one embodiment, third opening 314 has a diameter ranging between 2-5 mm.

First opening 310 is fluidly connected to distal end 106 of lamprey lock device 100 as described elsewhere herein. In one embodiment, first opening 310 has the same diameter as lumen 108 of lamprey lock device 100.

Second opening 312 is fluidly connected to a drainage reservoir. In one embodiment, the fluid connections can be any suitable mechanism known in the art configured to fluidly join tubes end-to-end, including but not limited to luer locks, tube fittings, threaded connectors, barbed connectors, and the like.

Body 306 further comprises a side port 316. Side port 316 is fluidly connected to body 306 through third opening 314. In one embodiment, side port 316 has an angle ranging between 20-90 degrees with respect to body 306.

Side port 316 comprises a first end 318, a second end 320 and a lumen therebetween. Second end 320 comprises an opening 322. In one embodiment, opening 322 may have the same diameter as third opening 314. In one embodiment, opening 322 may have a larger diameter as third opening 314. In one embodiment, opening 322 may have a smaller diameter than third opening 314. In one embodiment, opening 322 is sized and configured to connect to standard sizing for luer connectors, such as those that meet ANSI standards. In one embodiment, opening 322 may be fluidly connected to a syringe 323.

Side port 316 further comprises a valve member configured to allow selective fluid communication between body 306 and side port 316, wherein the valve member comprises a compressible component 324, a support structure 326 and at least one channel 328.

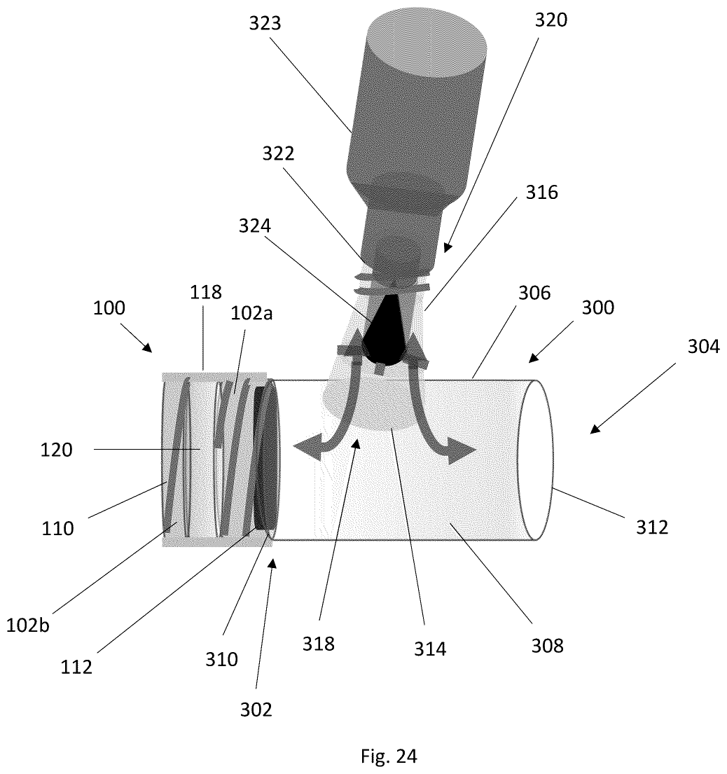

Compressible component 324 is configured to receive the luer tip of syringe 323 at second end 320 (FIG. 24). Once syringe 323 is inserted, the luer tip pushes compressible component 324 down which in turn causes at least one channel 328 to open and allow fluid communication between side port 316 and body 306. Once syringe 324 is unplugged, compressible component 324 springs back to its original position, such that a fluid tight seal is once again formed at the at least one channel 328.

Side port 316 further comprises a support structure 326. Support structure 326 is configured to guide compressible component 324 through its travel. In one embodiment, support structure 326 maintains proper alignment of compressible component 324 within the valve member which ensures leak free flow performance.

In one embodiment, body 306 and side port 316 may be constructed from a flexible plastic material. In one embodiment, body 306 may be made from a polymeric material, such as silicone, nylon, or urethane. Any medically acceptable thermoplastic or thermoset material may be used, including PTFE, a fluoropolymer, polyethylene, polypropylene, acetal, urethane, and others, however, it may be constructed from any material known to one skilled in the art. In one embodiment, body 306 may be made from a flexible material so that it is bendable throughout its length and at least at first opening 310, second opening 312 and third opening 314. In one embodiment, side port 316 may be made from a flexible material so that it is bendable throughout its length.

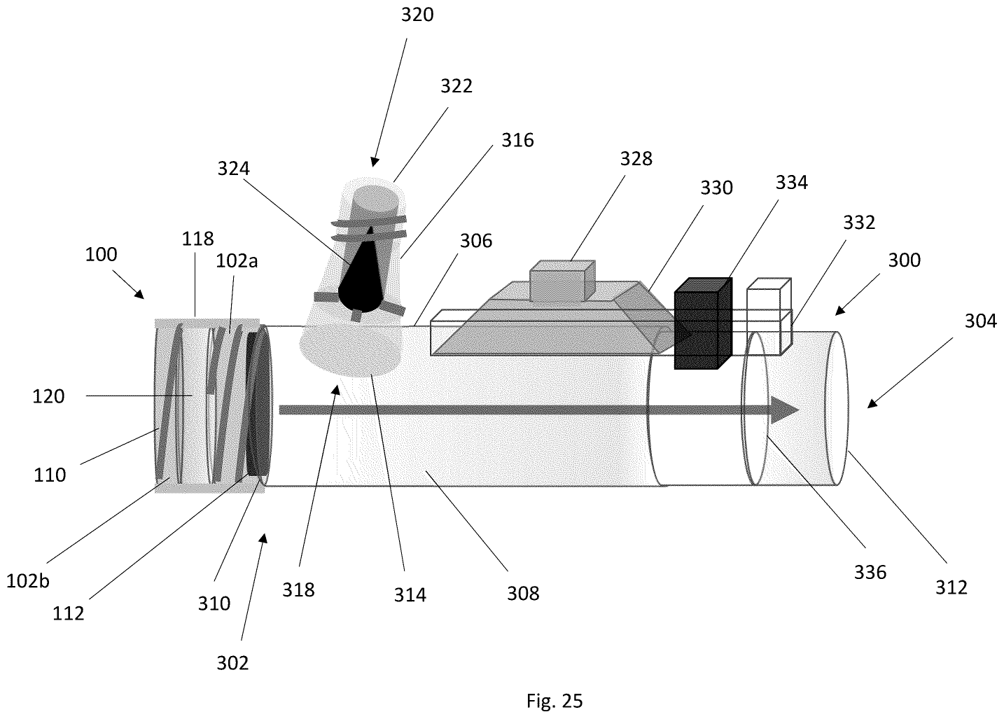

In one embodiment, tubing 300 may further comprise a flow switch 328 positioned on the external surface of body 306, configured to allow selective fluid communication between body 306 and drainage reservoir (FIG. 25). In one embodiment, flow switch 328 is positioned distal to side port 316.

Flow switch 328 comprises a tab 330, a track 332 configured to allow longitudinal movement of tab 330 between an open and close position, an occluding member 334 configured to move vertically and a compressible tubing 336.

Tab 330 may have a wide variety of shapes as known to one skilled in the art, including but not limited to cylindrical, prism-shaped, trapezoidal, square, or rectangular bars or beams, I-beams, elliptical beams, bowl-shaped surfaces, and others.

In operation, tab 330 is configured to move between an open and close position by slidably moving left and right on track 332. As tab 330 slides to the right on track 332, occluding member 334 is moved down vertically and pushes on compressible tubing 336. Applying a force to compressible tubing 336 causes flow restriction between body 308 and the drainage reservoir.

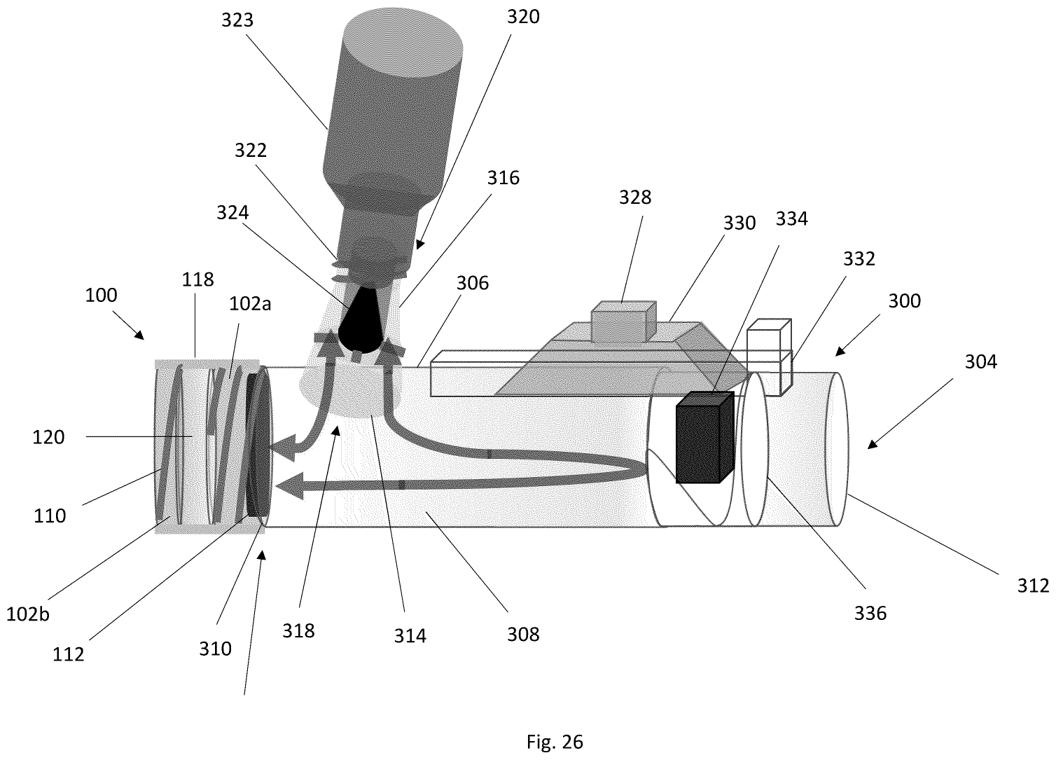

In one embodiment, if tab 330 is moved all the way to the right, occluding member 334 is pushed down completely, such that the fluid communication between body 306 and drainage reservoir is completely closed (FIG. 26). In one embodiment, tab 330 location may be placed anywhere in between the open and close position which in turn causes partial flow communication between body 306 and the drainage reservoir.

In one embodiment, compressible tubing 336 may be attached to distal end 304 of body 306. In one embodiment, compressible tubing 336 may have a length of approximately 4-4.5 mm. In one embodiment, compressible tubing 336 may be made from a polymeric material, such as silicone, nylon, or urethane. Any medically acceptable thermoplastic or thermoset material may be used, including PTFE, a fluoropolymer, polyethylene, polypropylene, acetal, urethane, and others, however, it may be constructed from any material known to one skilled in the art. In one embodiment, compressible tubing 336 may be made from any flexible material so that it is bendable throughout its length. In one embodiment, body 306 may be attached to compressible tubing 336 by any suitable mechanism known in the art configured to fluidly join tubes end-to-end, including but not limited to luer locks, tube fittings, threaded connectors, barbed connectors, and the like.

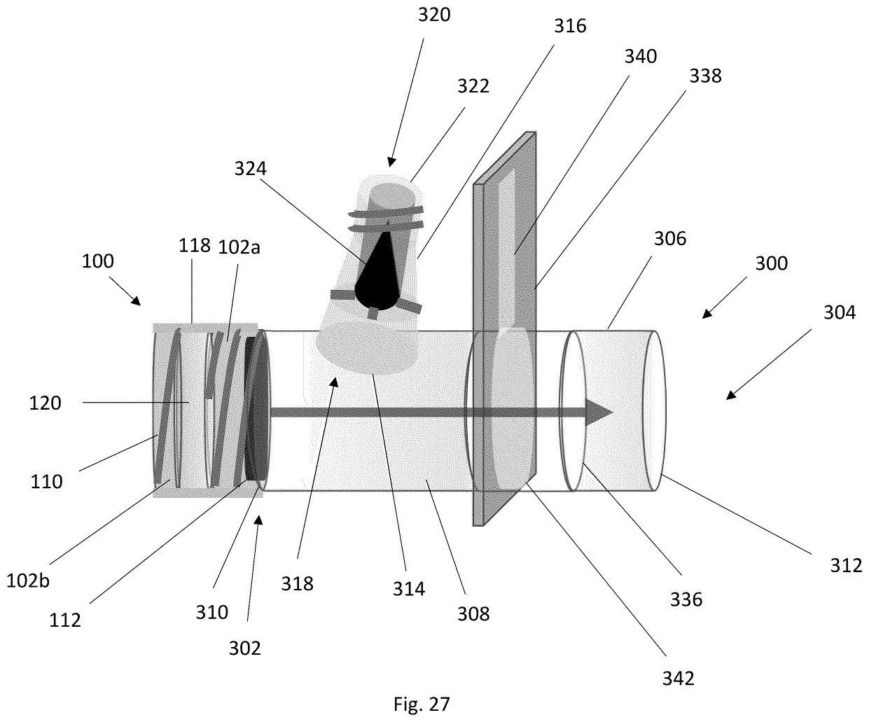

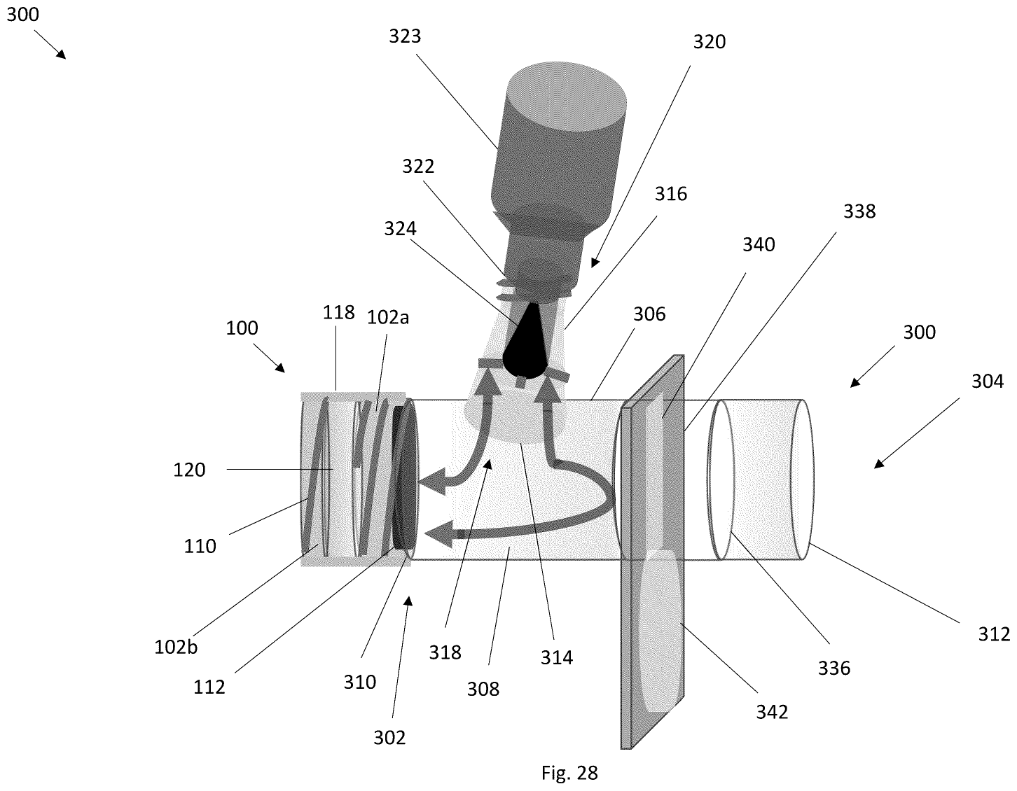

In one embodiment, tubing 300 may further comprise an external clamp 338 (FIG. 27) positioned distal to side port 316. External clamp 338 is connected to compressible tube 336 and thereby control the cross-sectional size of an opening in body 306, through which the fluid flows. External clamp 338 comprises a rounded opening 340 and an occluded slot 342. Rounded opening 340 has a diameter approximately equal or very slightly smaller than an exterior diameter of compressible tube 336. In one embodiment, the position of external clamp 338 along compressible tube 336 is adjustable by sliding external clamp along the length of compressible tube 336, while compressible tube 336 is located in rounded opening 340. The close size relationship of rounded opening 340 and the diameter of compressible tube 336 frictionally maintains external clamp 338 at a desired position along a length of compressible tube 336. When in rounded opening 340, compressible tube 336 is not occluded to inhibit the flow of fluid. Occluding slot 340 converges to create a narrow width into which compressible tube 336 is forced as shown in FIG. 28. When compressible tube 336 is forced into occluding slot 340, the sidewall of compressible tube 336 is pinched together, thereby occluding compressible tube 336 and preventing the flow of fluid from body 306 towards the drainage reservoir.

In one embodiment, external clamp 338 is a slide clamp. In one embodiment, external clamp 338 is a ramp or roller clamp. In on embodiment, external clamp 338 may be any other clamping mechanism known to one skilled in the art.

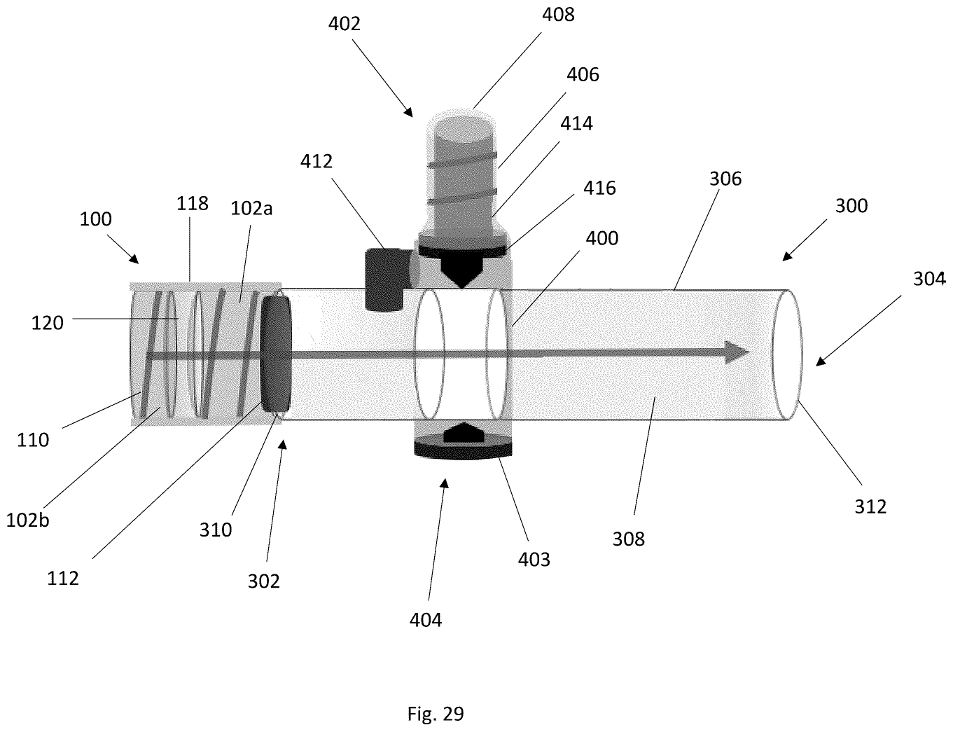

Referring now to FIG. 29, in one embodiment, tubing 300 may further comprise a chamber 400 positioned anywhere between first opening 310 and second opening 312, configured to engage the exterior surface of body 306.

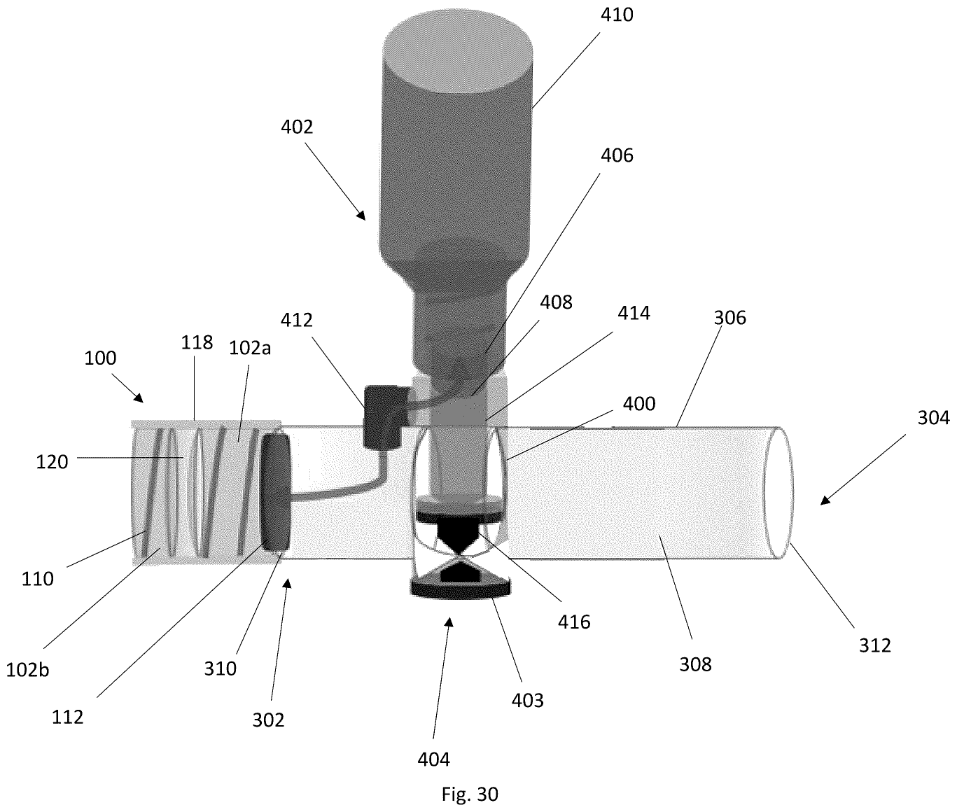

Chamber 400 comprises a first end 402, a second end 404, a side port 406 positioned at first end 402 and a bottom part 403 positioned at second end 404. In one embodiment, side port 406 comprises an opening 408 at first end 402. In one embodiment, opening 408 is sized and configured to connect to standard sizing for luer connectors, such as those that meet ANSI standards. In one embodiment, opening 408 may be fluidly connected to a syringe 410 (FIG. 30).

Side port 406 is fluidly connected to body 306 through a side channel 412. In one embodiment, side channel 412 has a diameter ranging between 2-5 mm. In one embodiment, side channel 412 may be any shape including but not limited to a L-shape tubing.

Side port 406 further comprises a valve member configured to allow selective fluid communication between body 306 and side port 406, wherein the valve member comprises a compressible component 414 and a tip 416 positioned below compressible component 414. Compressible component 414 is configured to receive the luer tip of syringe 410. Once syringe 410 is inserted, the luer tip pushes compressible component 414 down on body 306 all the way to bottom part 403, which in turn causes side channel 412 to open and allow fluid communication between side port 406 and body 306 and blocks fluid communication between catheter at first opening 310 and the drainage reservoir at second opening 312. Once syringe 410 is unplugged, compressible component 414 springs back to its original position, such that a fluid tight seal is once again formed between side port 406 and body 306.

In one embodiment, tip 416 is a pointed tapered tip. In one embodiment, tip 416 is a pointed tapered tip of at least 45 degrees to allow improved compression. In one embodiment, bottom part 403 also comprises a tapered component. In one embodiment, bottom part 403 comprises a pointed tapered tip of at least 45 degrees.

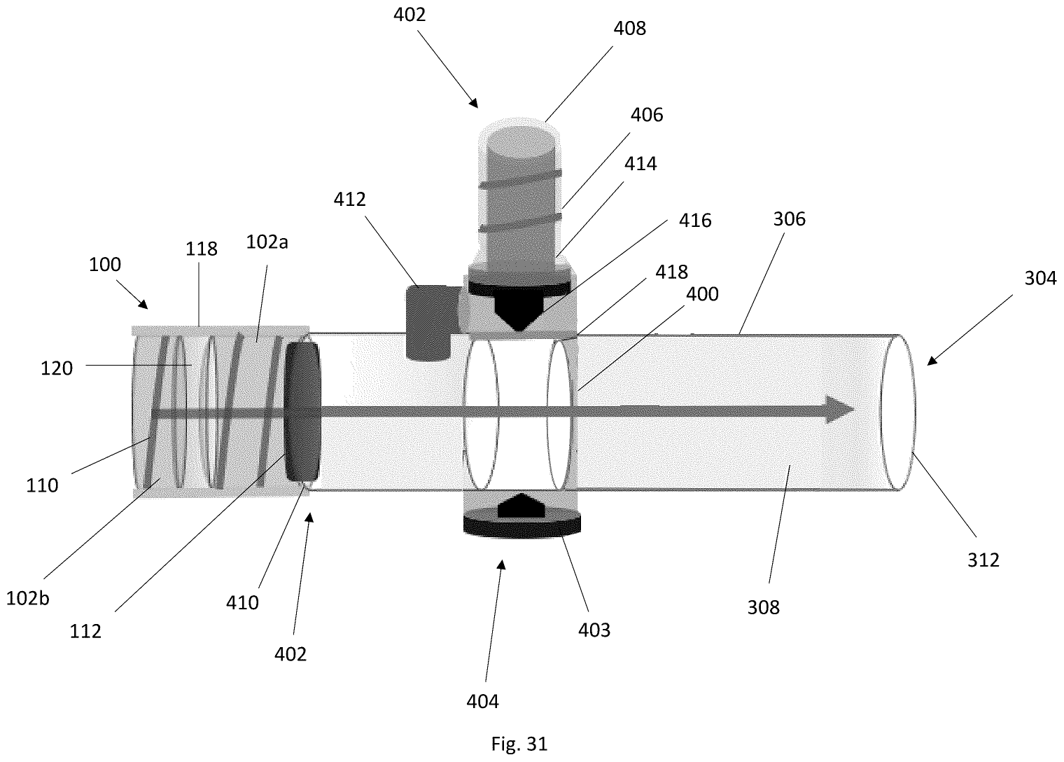

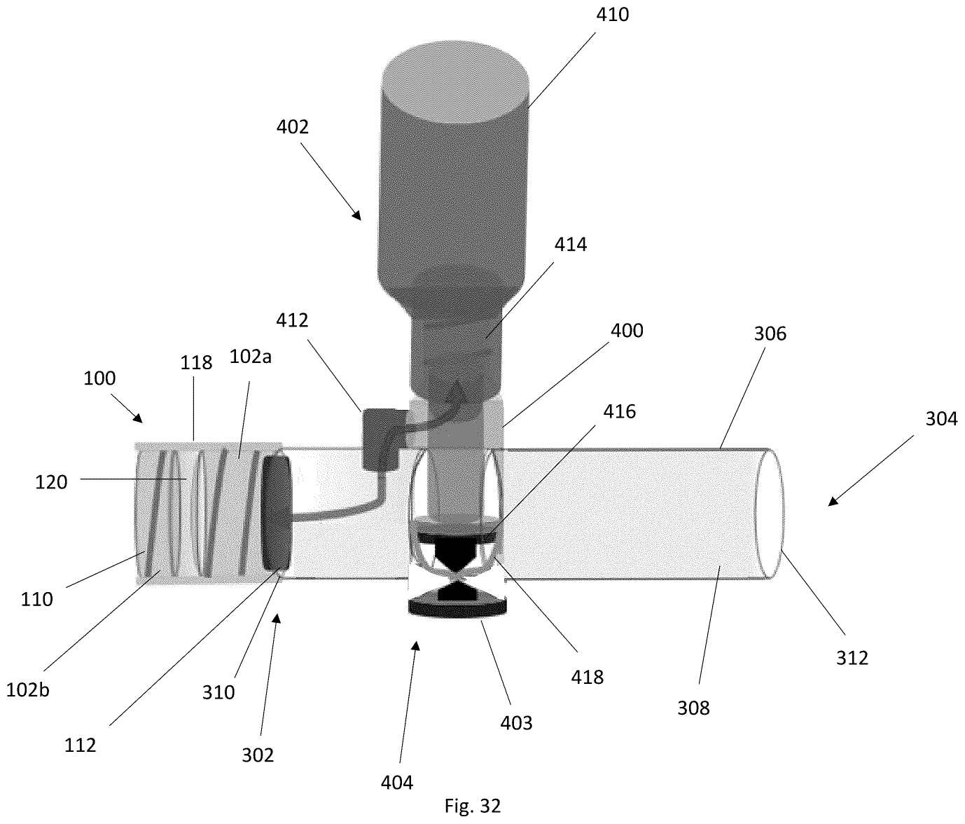

Referring now to FIG. 31 and FIG. 32, in one embodiment, chamber 400 further comprises a compressible membrane 418 positioned below tip 416 and over body 306. Compressible membrane 418 is configured to prevent fluid flow into inferior part of chamber 400 when valve member is in open position.

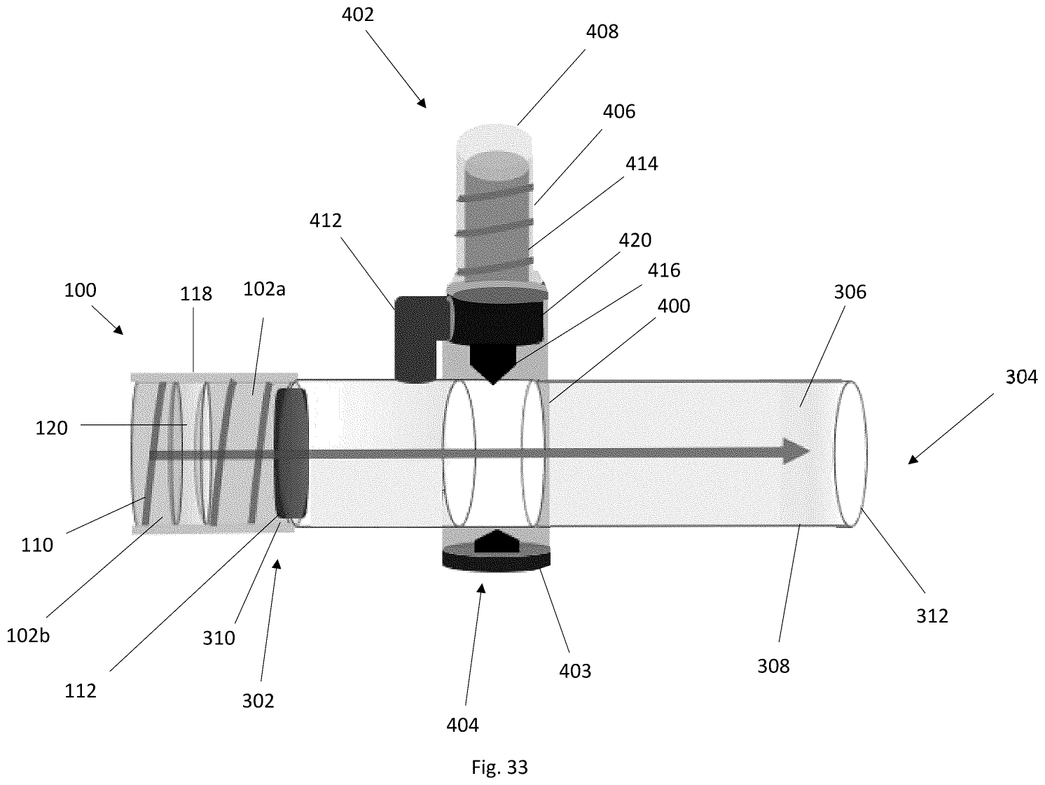

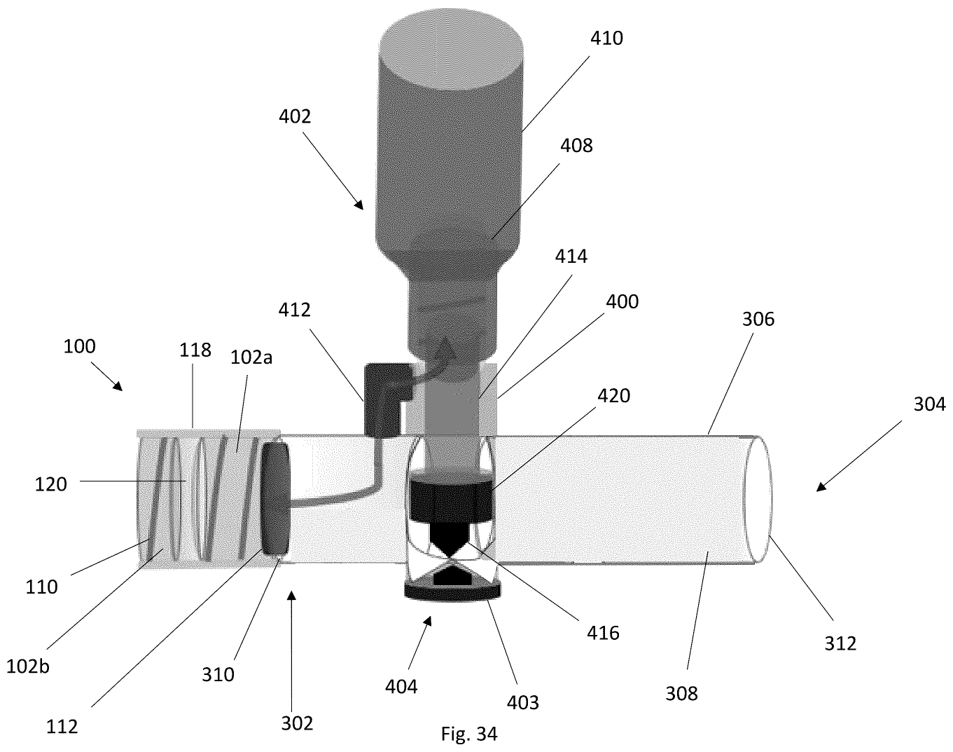

Referring now to FIG. 33, in one embodiment, tip 416 may comprise a base 420, configured to be wide enough to block flow into chamber 400 while in the closed position. Referring now to FIG. 34, once syringe 410 is inserted, the luer tip pushes compressible component 414 down on body 306 all the way to bottom part 403, which in turn causes base 420 to move down and unblocks the opening to side channel 412 and allow fluid communication between side port 406 and body 306 and blocks fluid communication between catheter at first opening 310 and the drainage reservoir at second opening 312.

### Method of Use

The present invention also relates to methods for fluid transfer between two devices or objects by maximizing the inner diameter of connections between two objects or devices including but not limited to a catheter, tubing, veress needles, trocars, syringes, or gas/fluid delivery systems. In one embodiment, the present invention also relates to methods of providing a connection between a catheter at its proximal end to a tubing including but not limited to a drainage reservoir tubing to allow flushing and draining fluid from a body cavity.

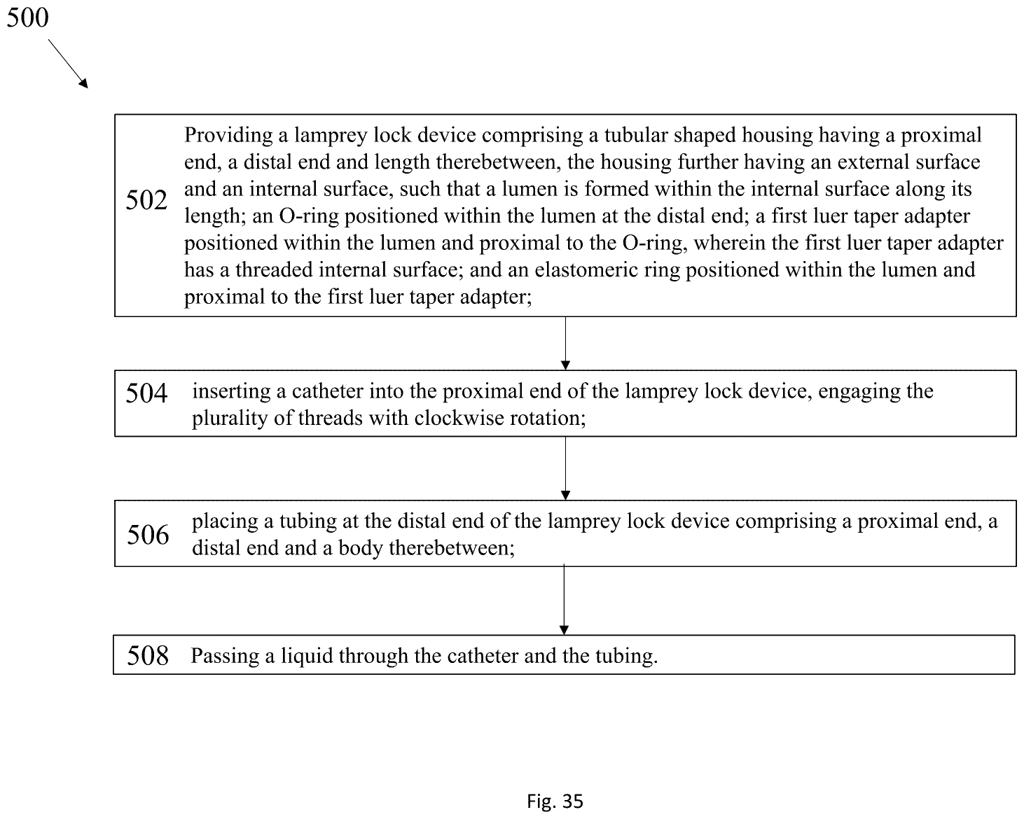

Referring now to FIG. 35, an exemplary method 500 of draining a body cavity is depicted. Method 500 begins with steps 502, wherein a lamprey lock device comprising a luer taper adapter having a proximal end, a distal end and a lumen therebetween, wherein the lumen comprises a plurality of threads within its interior wall; a circumferential elastomeric seal positioned at the proximal end of the luer taper adapter, configured to create a watertight seal with a proximal end of an inserted catheter; and a covering positioned around the luer taper adapter and the circumferential elastomeric seal, configured to secure the luer tap adapter and the circumferential elastomeric seal together is provided. In step 504, a catheter is inserted into the proximal end of the lamprey lock device, engaging the plurality of threads with clockwise rotation. In one embodiment, the clockwise rotation creates a watertight seal with the O-ring at the distal end. In step 506, a tubing is placed at the distal end of the lamprey lock device comprising a proximal end, a distal end, and a body therebetween. In step 508, a liquid is passed through the catheter and the tubing. In one embodiment, this connection may allow a liquid from a body cavity to be drained from the catheter to a drainage reservoir connected to the distal end of the tubing.

In one embodiment, the lamprey lock device of the present invention may be attached to a three-way stopcock. In one embodiment, the tubing may further comprise a side port fluidly connected to the body, wherein the side port comprises a first end, a second end and a lumen therebetween and is configured to be connected to standard sizing for luer connectors at second end and allow selective fluid communication between the body and the side port. In one embodiment, the tubing may further comprise a flow switch positioned on the external surface of the body and distal to the side port, configured to allow selective fluid communication between the body and the drainage reservoir. In one embodiment, the tubing may further comprise an external clamp positioned on the external surface of the body and distal to the side port, configured to allow selective fluid communication between the body and the drainage reservoir.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, make and utilize the compounds of the present invention and practice the claimed methods. The following working examples therefore specifically point out exemplary embodiments of the present invention and are not to be construed as limiting in any way the remainder of the disclosure.

### Example 1: Lamprey Lock Flow Data

### Experimental Setup

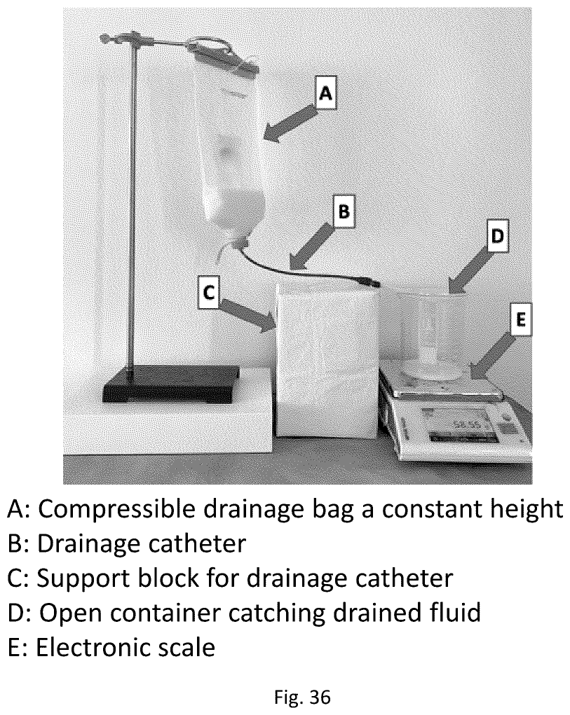

In vitro experiments simulated drainage of serous/purulent fluid (FIG. 36). In this experimental setup a 14 French drainage catheter (Total Abscession; Angiodynaics; Queensbury, NY) was inserted into a compressible reservoir. The distal female end of the drainage catheter was then connected to either a Luer connector or Lamprey lock. The reservoir was filled to an initial volume of 500 mL and was elevated above the catheter outlet to a constant fluid column height of 20 cm. Gravity drainage occurred into an open beaker atop an electronic balance (Precision Balances Model ML802T/00; Mettler-Toledo, Greifensee, Switzerland). Mass and time were simultaneously recorded at fixed intervals for a specified duration to determine mean drainage rate, total drainage time, and cumulative drainage or percentage of total volume drained. To calculate drainage rate, units of mass were converted to volume by dividing mass by specific gravity. Each experimental condition was repeated 5 times and results were averaged across trials and the mean performance of Luer and Lamprey lock connectors were compared.

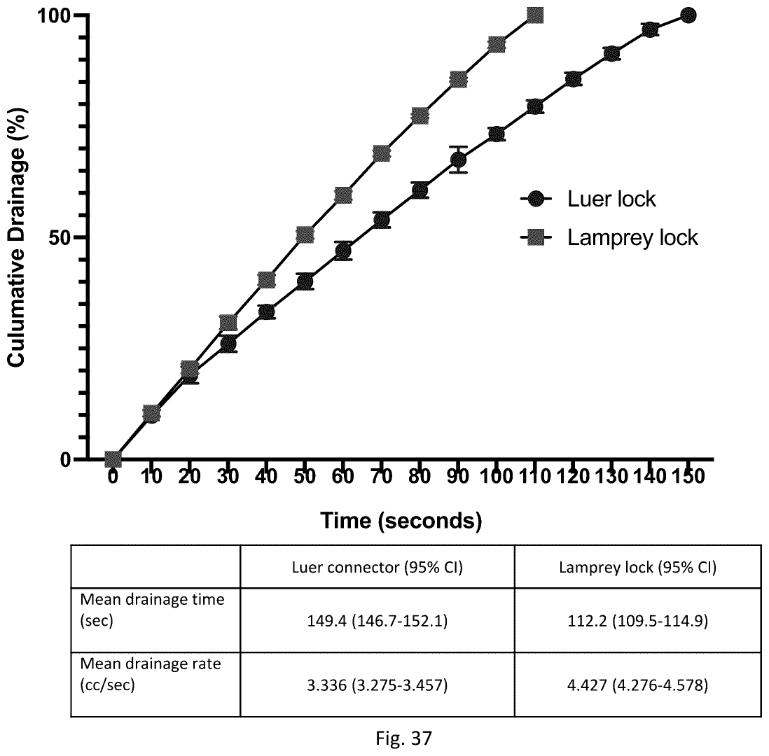

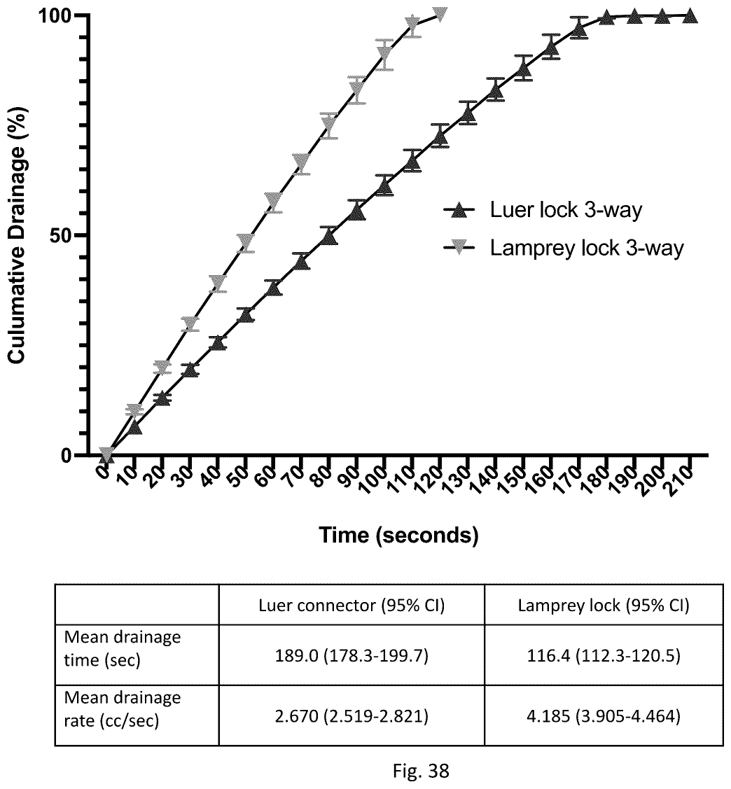

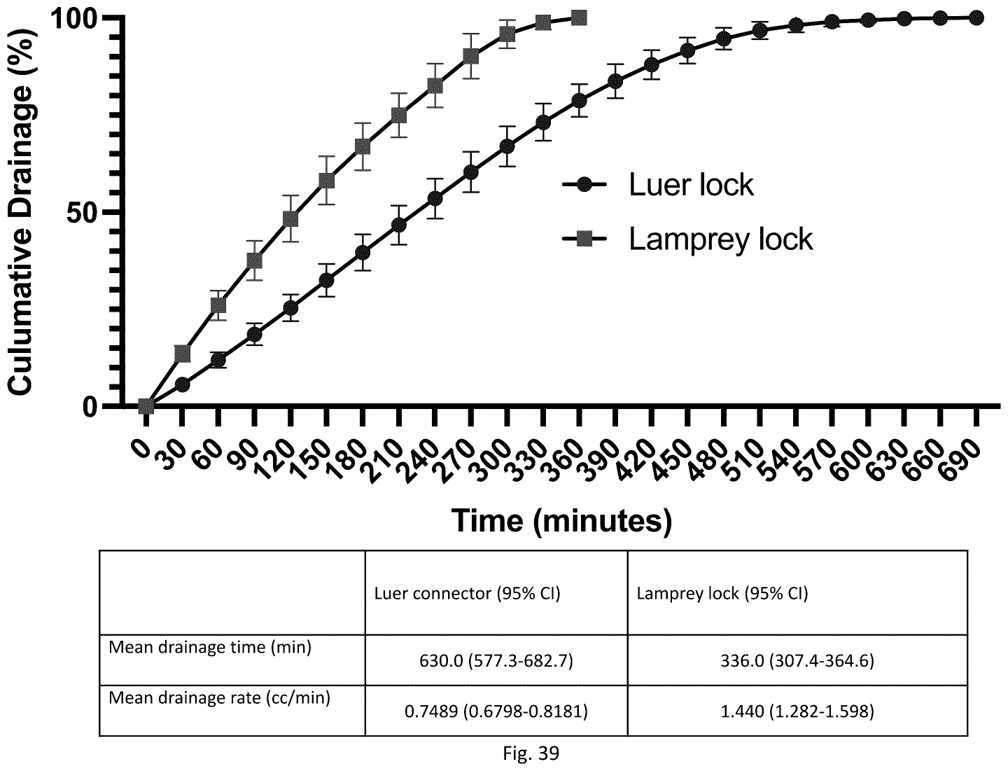

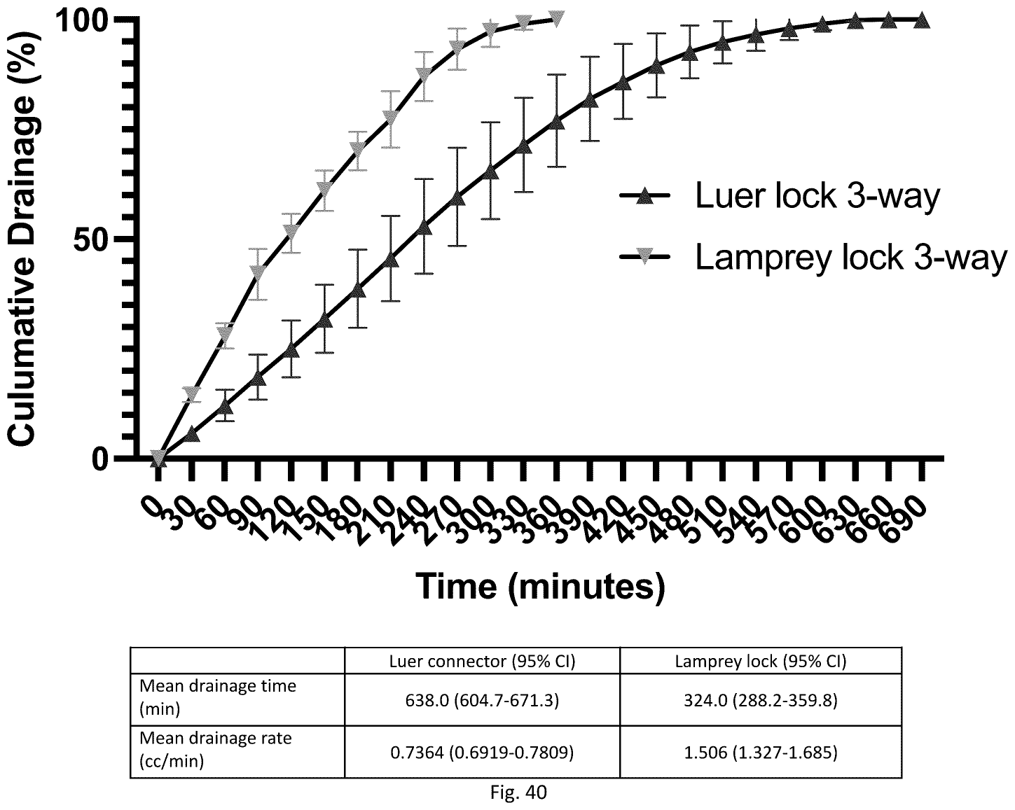

In the serous model, the Lamprey lock demonstrated a drainage rate 32.7% faster than the Luer lock (FIG. 37) and the 3-way Lamprey lock drainage rate was 56.7% faster than the 3-way Luer lock (FIG. 38). In the abscess model, the Lamprey lock has a drainage rate 92.3% faster than the Luer lock (FIG. 39) and the 3-way Lamprey lock drainage rate is 104.5% faster than the 3-way Luer lock (FIG. 40).

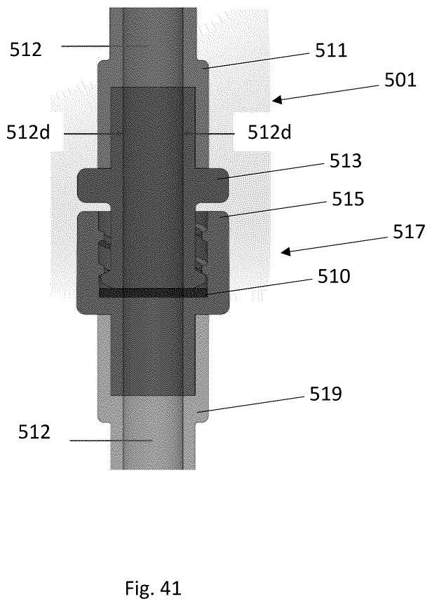

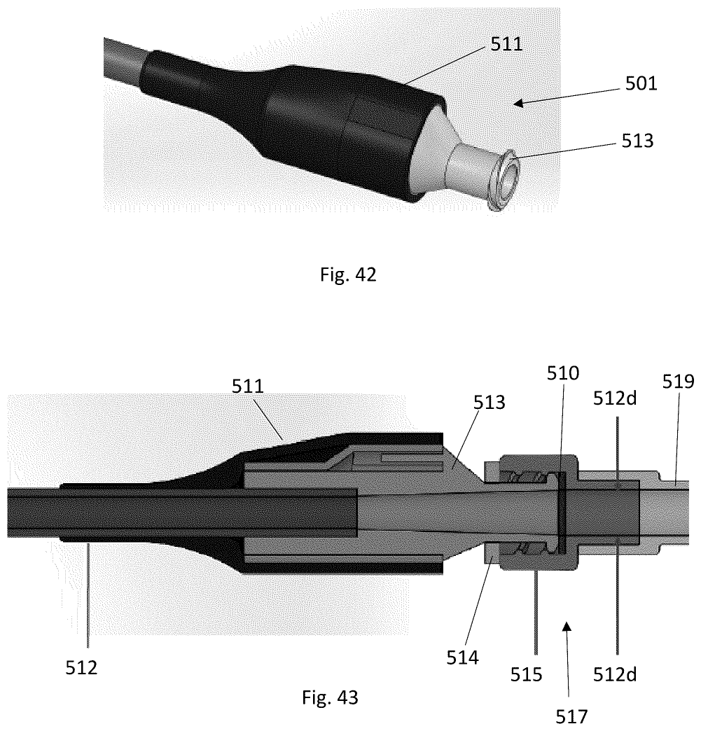

Referring now to FIG. 41, an exemplary lamprey lock device 517 of the present invention and an exemplary catheter 501 are shown. Lamprey lock device 517 may be coupled with a catheter 501. For instance, catheter 501 may have a housing 511 in which a Luer fitting 513 protrudes from the housing 511. The Luer fitting 513 may be a male or female fitting. The Luer fitting 513 may be sized to fit within a portion of the housing 515 of the lamprey lock device 517. An end of luer fitting 513 may contact a seal 510 positioned within the housing 515. The seal 510 may be, for example, an O-ring to prevent leaks in the connection of the catheter 501 and lamprey lock device 517. The seal 510 may be formed from material, such as, but not limited to, Buna-N rubber, silicone, ethylene propylene (EPDM), viton fluoroelastomer (FKM), Kalrez 7075, or other like materials. The lamprey lock device 517 may include the housing 515 coupled with a fitting 519. The fitting 519 may be any type of male or female luer fitting. The housing 515 and fitting 519 may be co-molded to one another. In some examples, the fitting 519 may be operably coupled with a collection bag to receive fluid passing from the catheter 501. The lamprey lock device 517 may be formed from a rigid or semi-rigid material, such as, but not limited to ABS, polycarbonate, polypropylene, nylon acrylic, styrene acrylonitrile resin, and other like materials. When catheter 501 and lamprey lock device 517 are coupled with one another, a lumen 512 is formed therein. The inner diameter of the lumen 512 may be consistent through the catheter 501 and lamprey lock device 517. For example, the inner diameter of the lumen 512 may be 4.4 mm.

FIG. 43 depicts a cross-sectional view of the lamprey lock device 517 coupled with the catheter 501. A diaphragm 514 may be molded onto the housing 515 as described herein. In some cases, the diaphragm 514 may be molded onto the housing 515 via overmolding, insert molding, or other like methods. The diaphragm 514 may be formed from a material, such as, but not limited to, silicone, thermoplastic elastomer (TPE), thermoplastic polyurethane (TPU), thermoplastic vulcanizate (TPV), thermoplastic copolyester (TPC), or other like materials. In some cases, the diaphragm 514 is positioned on the insertion end of the housing 515. The diaphragm 514 includes an opening allowing the insertion end of the luer fitting 513 to pass through the opening of the diaphragm 514. The diaphragm 514 may be snuggly fit around the luer fitting 513 to seal the connection between luer fitting 513 and the housing 515.

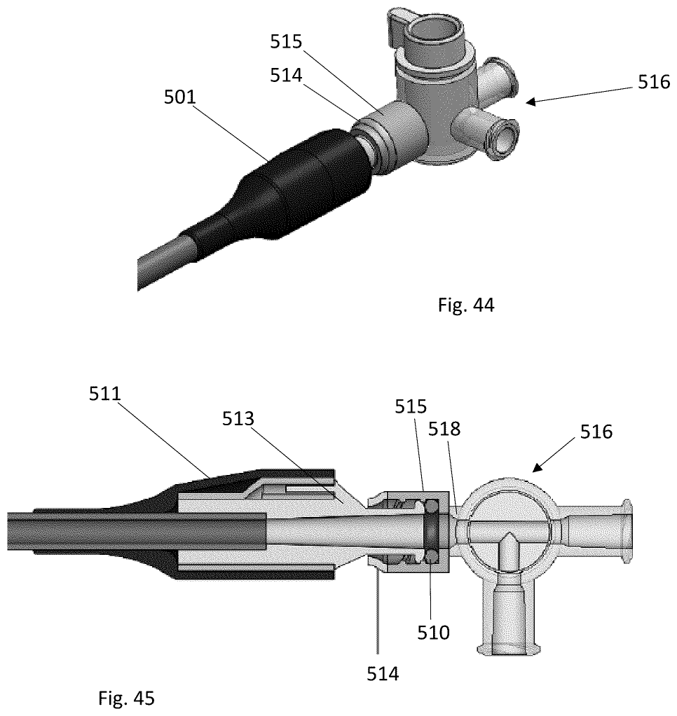

FIGS. 44 and 45 depict portions of the exemplary lamprey lock device integrated with a stopcock 516. For example, the housing 515 may be coupled to a port 518 of the stopcock 516. In some cases, diaphragm 514 is positioned on the insertion end of the housing 515. Further, the seal 510 may be provided within the housing 515. In some cases, the catheter 501 may be a pigtail catheter.

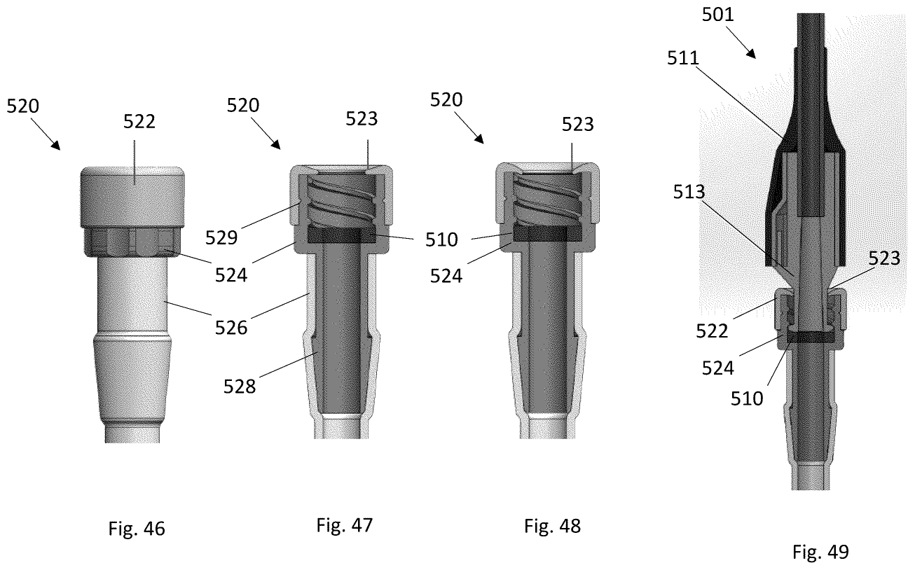

FIGS. 46-48 depict an exemplary lamprey lock device 520 having a three-part assembly. FIG. 49 illustrates a cross-sectional view of the lamprey lock device 520 coupled with the catheter 501. The lamprey lock device 520 includes one or more of the same or similar features as the lamprey lock device 517, and as such, a description of those features is not repeated. The lamprey lock device 520 includes a housing 524 coupled to a luer fitting 526. The housing 524 of the lamprey lock device 520 may be formed in the shape of the luer fitting 526. For example, the housing 524 may have a barb 528 or protruded area to match that of the luer fitting 526. The housing 524 includes a cavity sized to receive luer fitting 513 therein. In some cases, the interior wall that defines the cavity of the housing 524 may be threaded. In other cases, the interior wall may be substantially cylindrical and is not threaded. A seal 510 may be provided at the base of the cavity to provided a watertight seal between the luer fitting 513 and the housing 524. In some cases, the exterior surface of the housing 524 includes one or more gripping features disposed around the housing 524. In other cases, the exterior surface of the housing 524 is uniform and does not include gripping features. The lamprey lock device 520 includes a diaphragm 522 fitted over at least a portion of the housing 524. The diaphragm 522 may include a thin wiper seal 523 that defines an opening to receive the luer fitting 513 therein. In some cases, the seal 523 flexes inwards when receiving the luer fitting 513 and provides a snug fit around the luer fitting 513. In some cases, the housing 524 includes one or more grooves 529 provided on the exterior of the housing 524. A portion of the diaphragm 522 may reside within the one or more grooves 529 to positively interlock the diaphragm 522 to the housing 524.

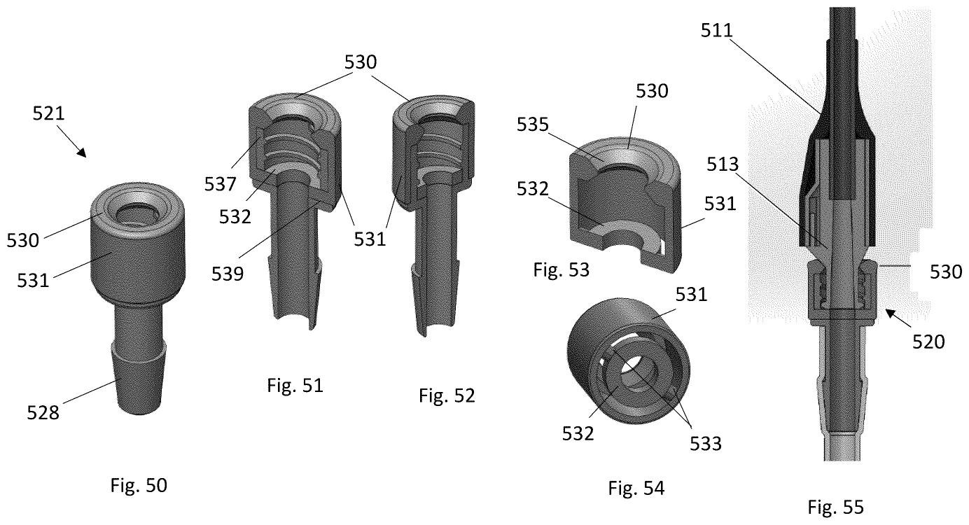

FIGS. 50-52 depict an exemplary lamprey lock device 521 having a three-part assembly. FIGS. 53 and 54 depict a diaphragm 530 of the lamprey lock device 521. FIG. 55 illustrates a cross-sectional view of the lamprey lock device 521 coupled with the catheter 501. The lamprey lock device 521 includes one or more of the same or similar features as the lamprey lock device 520 and lamprey lock device 517, and as such, a description of those features is not repeated.

The diaphragm 530 may include a seal 532 that is molded as one piece. The seal 532 may be, for example, an O-ring to prevent leaks in the connection of the catheter 501 and lamprey lock device 521. In some cases, the diaphragm 530 and seal 532 may be formed from the same material. In other cases, the diaphragm 530 and seal 532 may be formed from different materials. The seal 532 may operate in a same or similar manner as seal 510. The seal 532 may be coupled with the diaphragm 530 via one or more supports 533. The one or more supports 533 may provide a connection point between the wall 531 of the diaphragm 530 and seal 532 to form the diaphragm 530 and seal 532 as one mold. In some cases, the housing 537 of the lamprey lock device 521 includes one or more ports 539 that allow the one or more supports 533 to pass through to connect the wall 531 on the exterior of the housing 537 to the seal 532 positioned within the housing 537. In some cases, the diaphragm 530 may include a thin wiper seal 535 at the insertion end of the diaphragm 530, similar to that of the thin wiper seal 523 of the lamprey lock device 520. The wiper seal 535 may flex inwards and provide a snug fit around the luer fitting 513 of the catheter 501. The

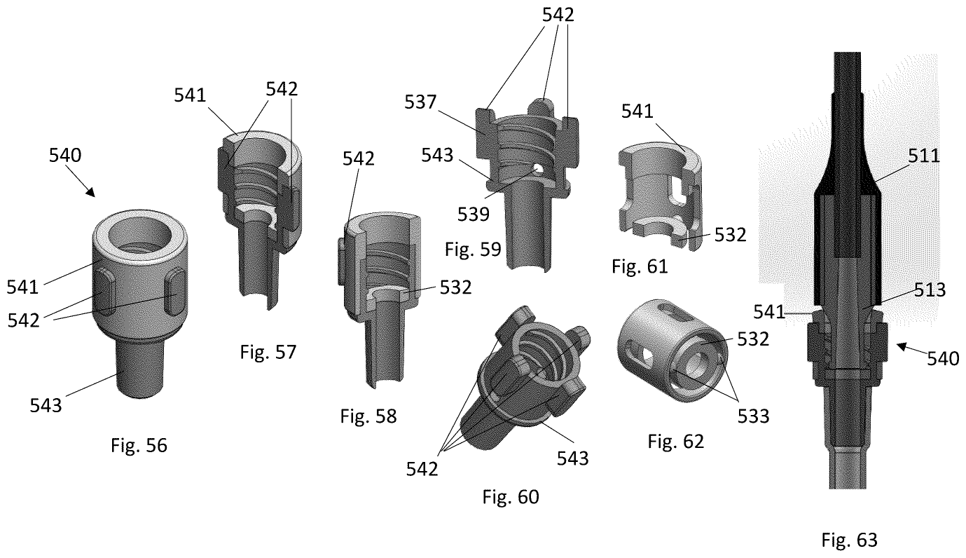

FIGS. 56-58 depict an exemplary lamprey lock device 540 having a three-part assembly. FIGS. 59 and 60 depict a housing 543 of the lamprey lock device 540. FIGS. 61 and 62 depict a diaphragm 541 of the lamprey lock device 540. FIG. 63 illustrates a cross-sectional view of the lamprey lock device 540 coupled with the catheter 501. The lamprey lock device 540 includes one or more of the same or similar features as the lamprey lock devices 517, 520, and 521, and as such, a description of those features is not repeated.

Similar to that of lamprey lock device 521, the seal 532 may be molded with the diaphragm 541 via one or more supports 533. The housing 543 may include one or more protrusions 542 disposed around the exterior surface of the housing 543 The one or more protrusions 542 may extend outwards from the surface of the housing 543. The diaphragm 541 may be formed around the one or more protrusions 542 such that the diaphragm 541 and housing 543 are positively interlocked with one another.

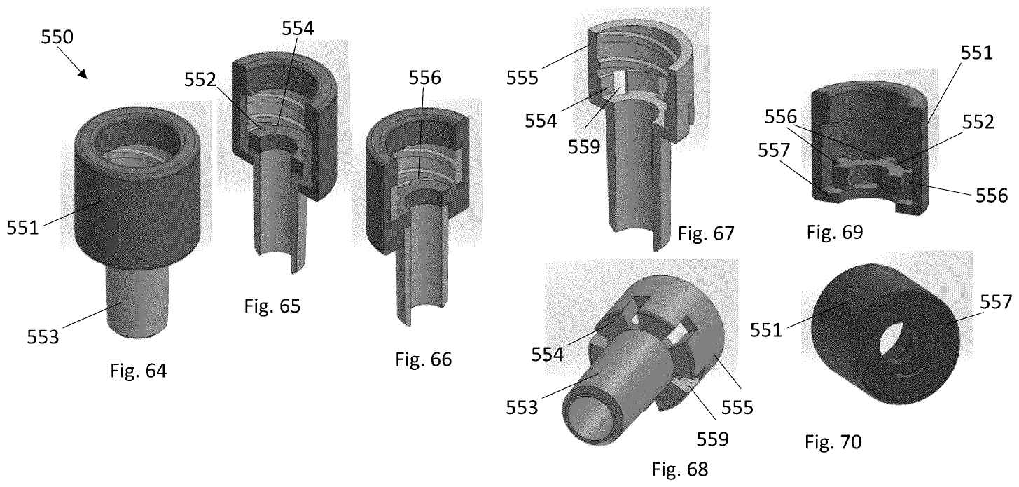

FIGS. 64-66 depict an exemplary lamprey lock device 550 having an overmolded seal 552 and a diaphragm 551. FIGS. 67 and 68 depict a housing 553 of the lamprey lock device 550. FIGS. 69 and 70 depict the diaphragm 551 of the lamprey lock device 550. The lamprey lock device 550 includes one or more of the same or similar features as the lamprey lock devices 517, 520, 521, and 540 and as such, a description of those features is not repeated. The housing 553 includes a body 555 having a series of members 554 that define one or more openings 559 within a wall of the housing 553. The diaphragm 551 may be, for example, a grip-diaphragm-delete, in that the diaphragm 551 is molded around the entire outer surface of the body 555. The base 557 of the diaphragm 551 may be positioned on the bottom of the body 555. The seal 552 of the diaphragm 551 may be positioned within the housing 553 at the base of the body 555. The seal 552 may perform the same or similar functions as seal 510. The seal 552 and diaphragm 551 may be molded as one body. The seal 552 and diaphragm 551 may be coupled with one another via one or more support members 556. The one or more support members 556 may be formed through the one or more openings 559 of the housing 553 during the molding process. As such, the diaphragm 551 is positively interlocked with housing 553. The lamprey lock device 550 may provide a reduced diaphragm 551 that is over-molded without an undercut.

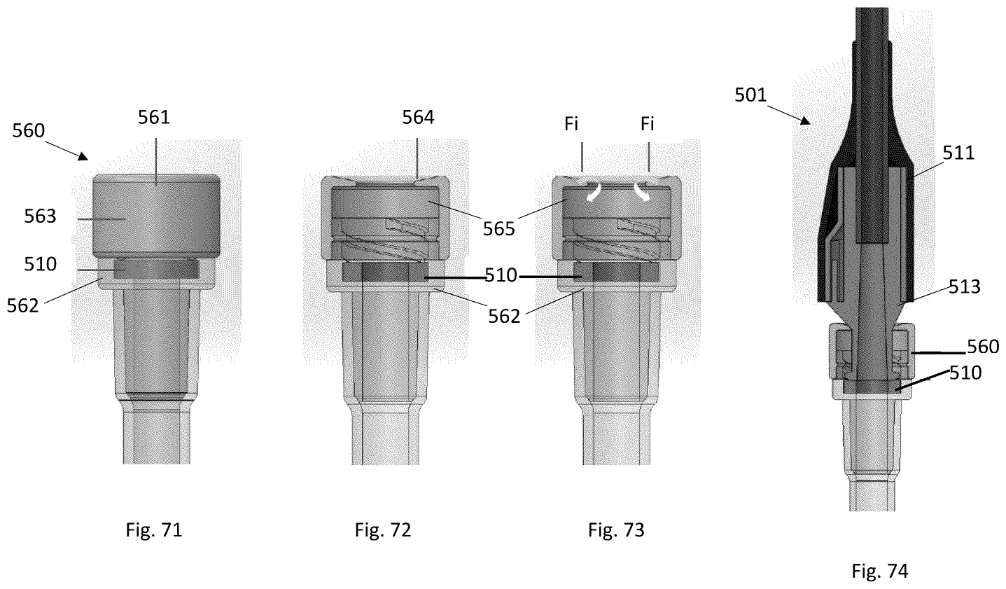

FIGS. 71-73 depict an exemplary lamprey lock device 560 having a three-part assembly. FIG. 74 illustrates a cross-sectional view of the lamprey lock device 560 coupled with the catheter 501. The lamprey lock device 560 includes one or more of the same or similar features as the lamprey lock devices 517, 520, 521, 540, and 550, and as such, a description of those features is not repeated.

The lamprey lock device 560 includes a housing 562 and a diaphragm 561. The diaphragm 561 is fitted over the housing 562 in a same or similar manner as lamprey lock device 520. The body 563 of the diaphragm 561 may surround at least a portion of the housing 562. The diaphragm 561 may be formed from a material, such as, but not limited to, TPU. The TPU material may have a 80A durometer. In some cases, the diaphragm 561 includes a thin wiper seal 564 and an overmolded portion 565. The seal 564 may define an opening to receive the luer fitting 513 therein. The seal 564 is configured to flex inward in direction Fi as the luer fitting 513 passes through the opening defined by the seal 564. The portion 565 of the diaphragm 561 may reside within a portion of the cavity of the housing 562. The seal 510 may reside within the cavity of the housing 562 and interfaces with the luer fitting 513 to provide a leak free connection. The seal 510 may be formed from a material, such as, but not limited to, silicone. The silicone may have a 70A durometer. The size of the housing 562 that receives the luer fitting 513 may be reduced compared to that of lamprey lock device 520, such that the luer fitting 513 travels a shorter distance to be fully seated within the housing 562.

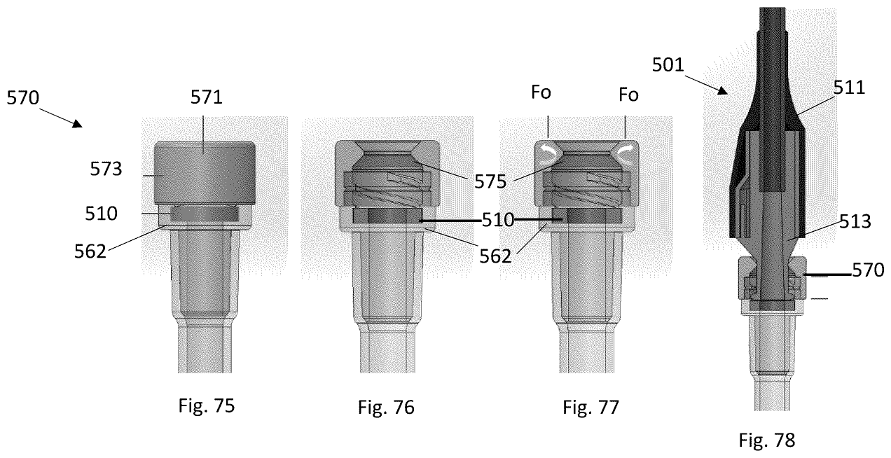

FIGS. 75-77 depict an exemplary lamprey lock device 570 having a three-part assembly. FIG. 78 illustrates a cross-sectional view of the lamprey lock device 570 coupled with the catheter 501. The lamprey lock device 570 includes one or more of the same or similar features as the lamprey lock devices 517, 520, 521, 540, 550, and 560, and as such, a description of those features is not repeated. In particular, the lamprey lock device 570 includes the same or similar features as lamprey lock device 560. However, the diaphragm 571 of the lamprey lock device 570 is distinguishable from the diaphragm 561 of the lamprey lock device 560 in that the seal 575 includes a ramp-in ramp-out shape. The seal 575 is configured to flex outwards in direction Fo as the luer fitting 513 is seated within the housing 562 of the lamprey lock device 570. The body 573 of the diaphragm 571 may be formed from a material, such as, but not limited to, an elastomer material. The elastomer material may have a 50A durometer.

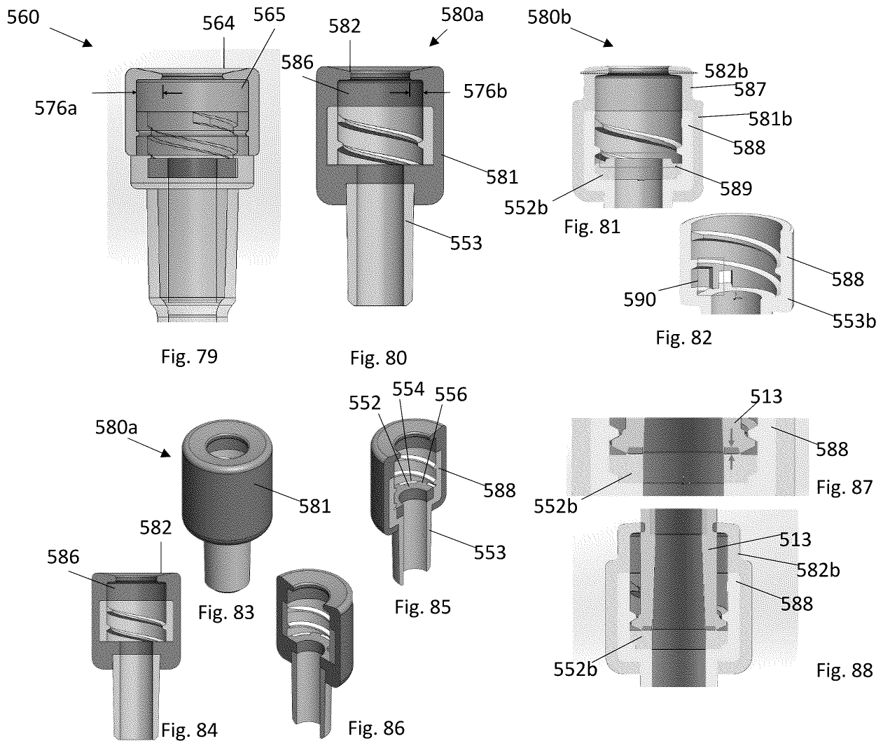

FIGS. 80 and 83-86 depict an exemplary lamprey lock device 580a having an overmolded seal 552 and a diaphragm 581 having a wiper seal 582 with a reduced undercut 576b. The seal 552 may be molded to the housing 553 in a same or similar manner as that described with respect to lamprey lock devices 550 and 560. The diaphragm 581 may include the thin wiper seal 582, which is similar to that of the seal 564 of diaphragm 561. However, the seal 582 may have a reduced undercut 576b on the overmolded portion 586 compared to that of the undercut 576a of the overmolded portion 565 of lamprey lock device 560. The reduced undercut 576b allows the lamprey lock device 580a to connect to catheters having a variety of sizes, even those with a short “neck”.

FIGS. 81 and 82 depict an exemplary lamprey lock device 580b having a diaphragm 581b with an end 587 having an inclined wiper seal 582b. The lamprey lock device 580b may include the same or similar features as lamprey lock device 580a. The lamprey lock device 580b is distinguishable from the lamprey lock device 580a in that the seal 582b may be inclined. The inclined surface of wiper 582b accommodates the retraction of the molding core. Additionally, the end 587 of the diaphragm 581b has a reduced outer diameter compared to that of the outer diameters of the diaphragm 551 or diaphragm 581. In some cases, the lamprey lock device 580b includes a seal 552b having a stepped portion 589. The stepped portion 589 increases interference with an end of the luer fitting 513 to ensure a proper seal, as illustrated in FIGS. 87 and 88. Further, the body 588 of the housing 553b may include a thread stop 590 within the interior of the body 588. The thread stop 590 ensures that the luer fitting 513 is stopped at a height within the body 588. When the luer fitting 513 is inserted into the body 588, the luer fitting 513 may be seated in the body 588 until the end of the luer fitting 513 contacts the thread stop 590. The luer fitting 513 is then compressed against the thread stop 590. A compression of 17.5% to 80.8% is achieved when the luer fitting 513 is pressed against the thread stop 590. As such, a user may rotate the luer fitting 513, for example, but not limited to, about 40% of the total rotation to achieve a minimum desired seal.

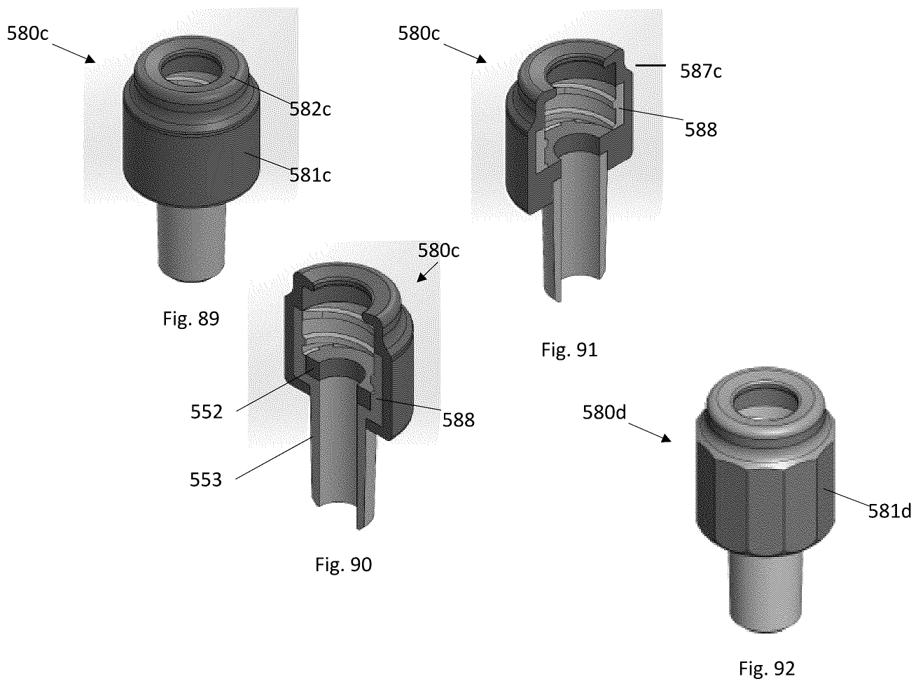

FIGS. 89-91 depict an exemplary lamprey lock device 580c having an overmolded seal 552 and a diaphragm 581c with an end 587 having a wiper seal 582c. The lamprey lock device 580c may include the same or similar features as lamprey lock device 580a and lamprey lock device 580b. The lamprey lock device 580c is distinguishable from lamprey lock device 580a in that end 587c of the diaphragm 581c has a reduced outer diameter compared to that of the outer diameters of the diaphragm 551 or diaphragm 581. The lamprey lock device 580c is distinguishable from lamprey lock device 580b in that the end 587c is curved and is more flexible than the end 587 of lamprey lock device 580b. FIG. 92 depict an exemplary lamprey lock device 580d that includes one or more of the same or similar features as lamprey lock device 580c. The lamprey lock device 580d may include one or more gripping features provided on the outer surface of the diaphragm 581d.

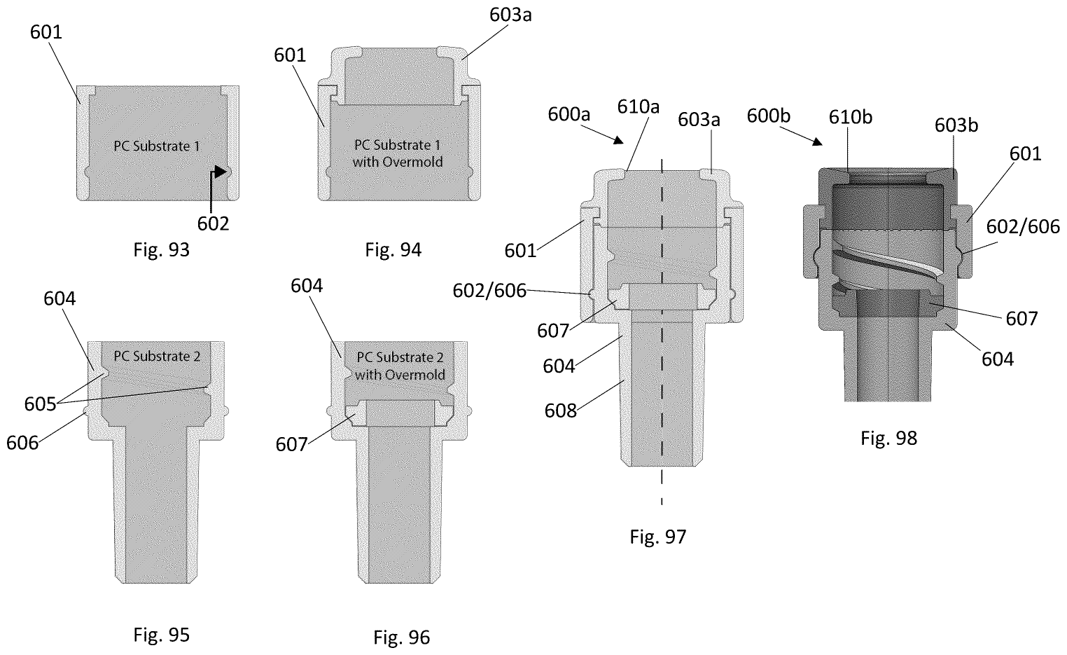

FIGS. 93 and 95 depict a top portion 601 and a bottom portion 604 of a housing 608 of an exemplary lamprey lock device 600a. FIG. 94 depicts an overmolded diaphragm 603 on the top portion 601 of the housing 608. FIG. 96 depicts a seal 607 on the bottom portion 604 of the housing 608. FIG. 97 depicts a cross-sectional view of an assembled exemplary lamprey lock device 600a having an overmolded diaphragm 603a and a seal 607. FIG. 98 depicts a cross-sectional view of an assembled exemplary lamprey lock device 600b having an overmolded diaphragm 603b and a seal 607.

The lamprey lock devices 600a and 600b include the same or similar features as lamprey lock devices 517, 520, 521, 540, 550, 560, 570, and 580 and as such, a description of those features is not repeated. The lamprey lock devices 600a and 600b are distinguishable from the other lamprey lock devices in that the lamprey lock devices, such as lamprey lock device 600a, may be manufactured as two separate components, such as the top portion 601 and the bottom portion 604, and subsequently snapped together to from the lamprey lock device 600a. Such manufacturing process and configuration of the lamprey lock devices allow the lamprey lock devices to be manufactured at higher volumes.

To manufacture the lamprey lock device 600a, the top portion 601 is formed, in which one or more dents 602 are formed in the inner sidewall of the top portion 601. The diaphragm 603a is molded onto the top surface of the top portion 601. The overmolded diaphragm 603a may include a wiper 610a, which may include any of the wiper shapes and/or features as described herein. The bottom portion 604 is formed, in which the inner surface of the bottom portion 604 is threaded to include threads 605 and one or more protrusions 606 are formed on the outer surface of the bottom portion 604. The seal 607 is provided within the bottom portion 604. For example, the seal 607 may be an O-ring that is inserted into the cavity of the bottom portion 604. In another example, the seal 607 is molded into the cavity of the bottom portion 604. The seal 607 may include any of the shapes or features of the seals described herein.

To assemble the lamprey lock device 600a, the top portion 601 is positioned over the bottom portion 604, and the one or more one or more protrusions 606 are positioned within the one or more dents 602, thereby locking the top portion 601 and bottom portion 604 to one another. The top portion 601 and the bottom portion 604 coupled to one another may form the housing 608 of the lamprey lock device 600a. It is noted that the lamprey lock device 600a is described as the top portion 601 includes one or more dents 602 and the bottom portion 604 includes one or more protrusions 606. However, it should be understood that the top portion 601 may include one or more protrusions 606 on the inner surface of the top portion 601, and that the 604 may include one or more dents 602 on the outer surface of the bottom portion 604.

It is noted that the lamprey lock device 600b includes the same or similar features as the lamprey lock device 600a. The lamprey lock device 600b is depicted to illustrate a lamprey lock device having a diaphragm 603b with an alternative wiper 610b, which may include any of the wiper shapes and/or features as described herein. Further, the top portion 601 may snap into the bottom portion 604, in which a portion of the overmolded diaphragm 603b forms a seal between the top portion 601 and bottom portion 604, thereby providing a secondary seal in addition to the seal provided by seal 607. In one or more cases, as an alternative to coupling the top portion 601 to the bottom portion 604, the top portion 601 of lamprey lock device 600a and lamprey lock device 600b may be used with a three-way valve.

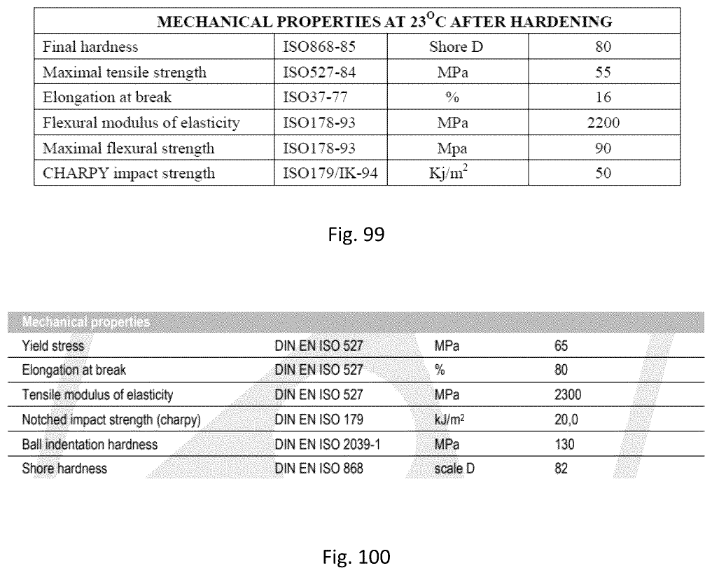

FIGS. 99 and 100 are tables describing mechanical properties of an example lamprey lock device, such as lamprey lock device 600a and lamprey lock device 600b.

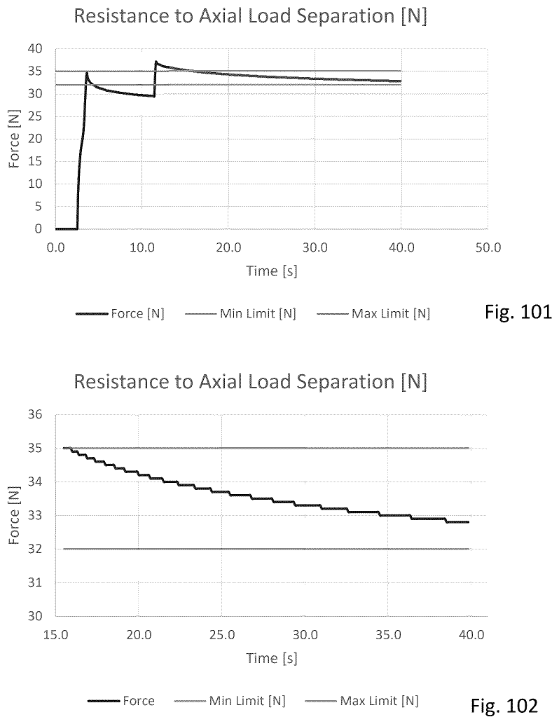

The example device was tested for resistance to axial load separation. The example device was tested against Luer slip connectors. As such, the example device and luer slip connectors were evaluated for separation from axial load. The example device and luer slip connectors were not separated from a reference connector over a hold period between 10 seconds and 15 seconds while being subjected to a disconnection applied axial force between. As illustrated in FIG. 101, the example device was subjected to a force of 32N to 35N, whereas the luer slip connector was subjected to a force of 23N and 25N.

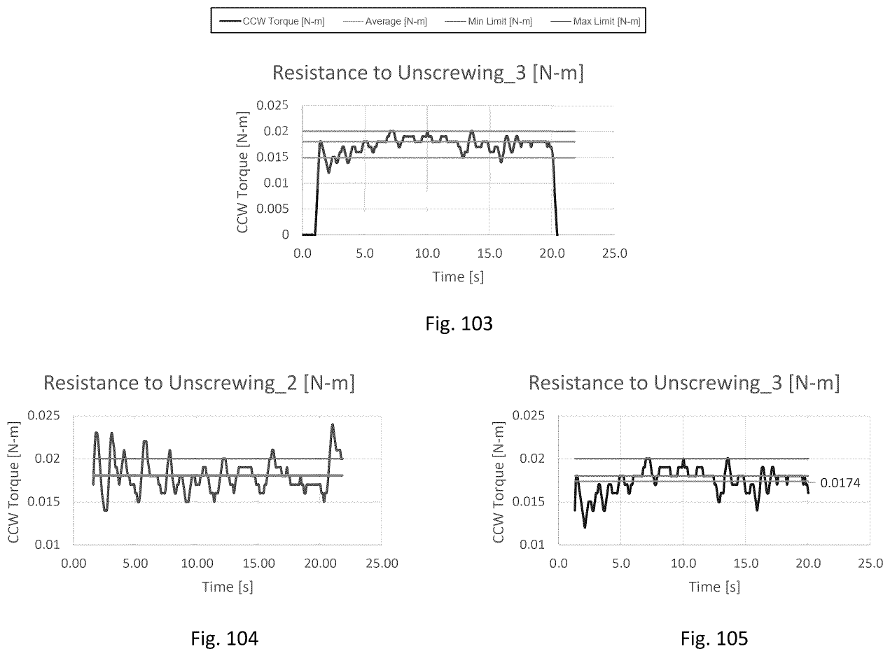

The example device was tested for resistance to unscrewing. The example device was evaluated for separation from unscrewing. The example device was not separated from the reference connector for a hold period between 10 s and 15 s while being subjected to an unscrewing torque of between 0,018 N·m to 0,020 N·m, as illustrated in FIGS. 103-105.

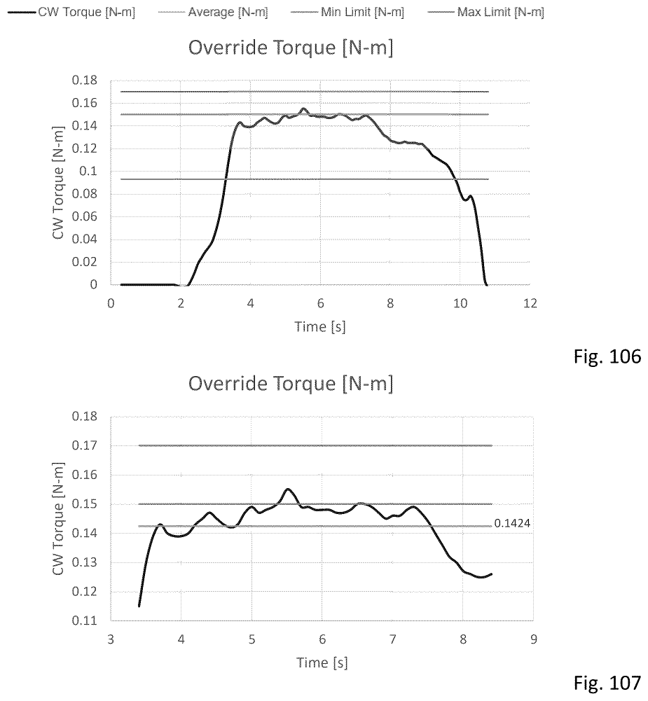

The example device was tested for resistance to overriding. The example device was evaluated for resistance to overriding. The example device did not override the threads or lugs of the reference connector while being subjected to an applied torque of between 0.15 N·m to 0.17 N·m over a hold period between 5 s and 10 s, as illustrated in FIGS. 106 and 107.

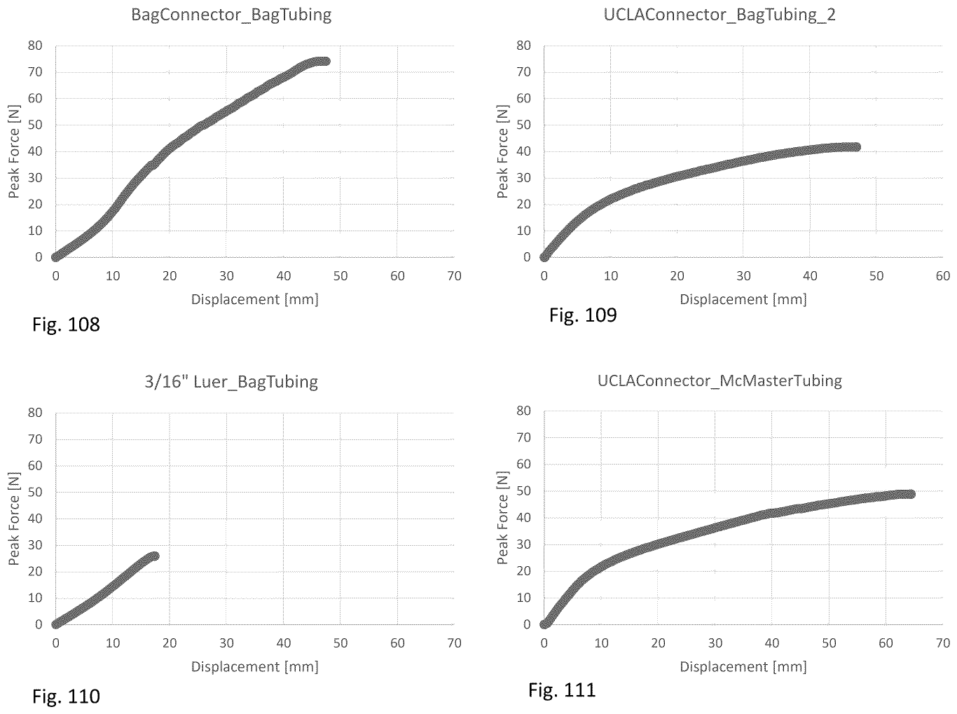

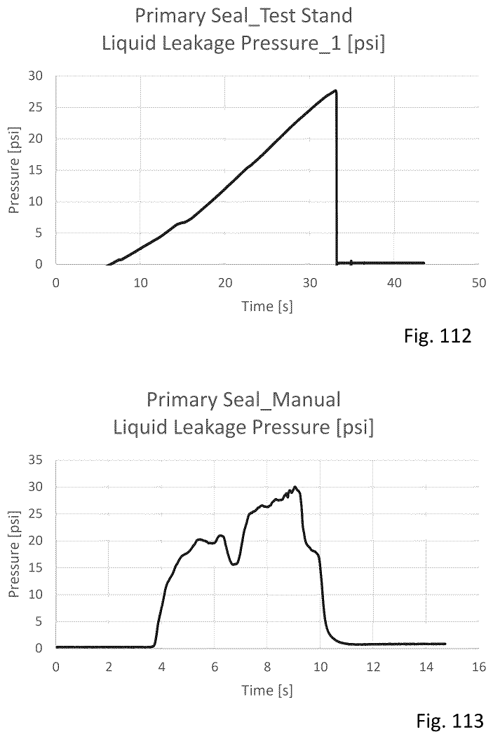

FIGS. 108-111 are tables illustrating the tubing detachment force of the example device from various connectors. FIGS. 112 and 113 are tables illustrating positive pressure liquid leakage testing of the example device. The example device was evaluated for fluid leakage performance with the positive pressure liquid leakage test method, which showed no signs of leakage, sufficient to form a falling drop of water, over a hold period of 30 s to 35 s while being subjected to an applied pressure of between 300 kPa and 330 kPa.

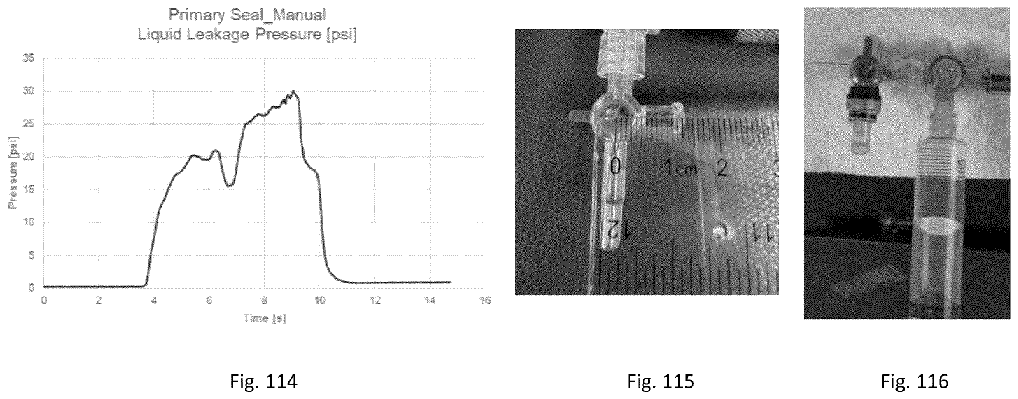

FIGS. 115 and 116 illustrate an initial pressure test setup for the lamprey lock device 600b. The lamprey lock device 600b was evaluated for fluid leakage performance with the positive pressure liquid leakage test method that showed no signs of leakage, sufficient to form a falling drop of water, over a hold period of 30 s to 35 s while being subjected to an applied pressure of between 300 kPa and 330 kPa, as illustrated in FIG. 114.

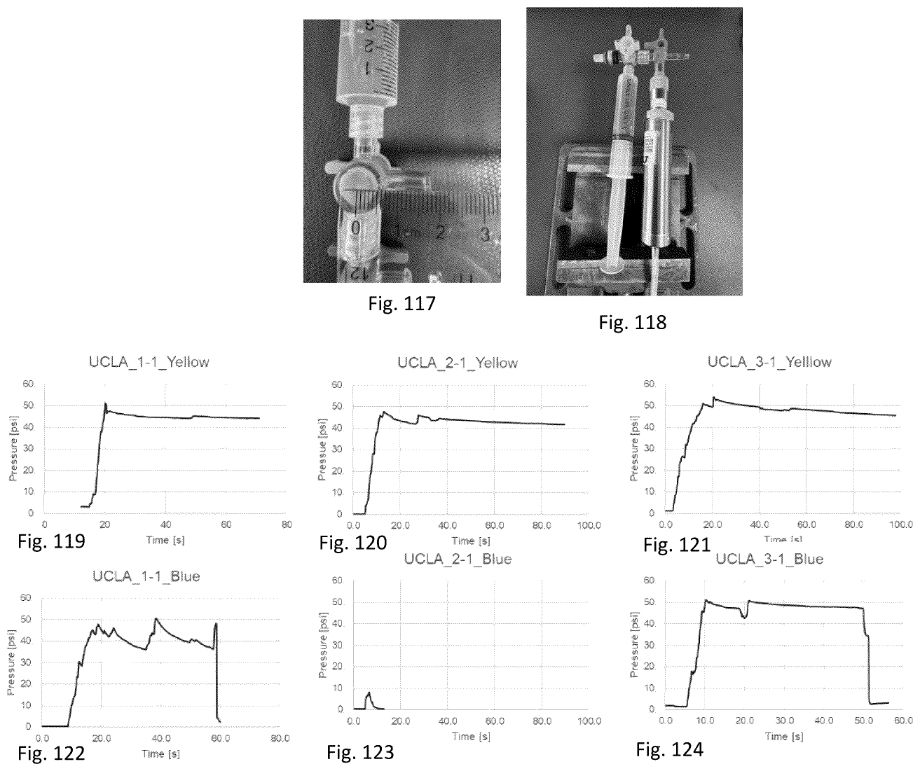

FIGS. 117 and 118 illustrate a secondary pressure test setup for the lamprey lock device 600b. The lamprey lock device 600b was evaluated for fluid leakage performance with the positive pressure liquid leakage test method that showed no signs of leakage, sufficient to form a falling drop of water, over a hold period of 30 s to 35 s while being subjected to an applied pressure of between 300 kPa and 330 kPa, as illustrated in FIGS. 119-124.

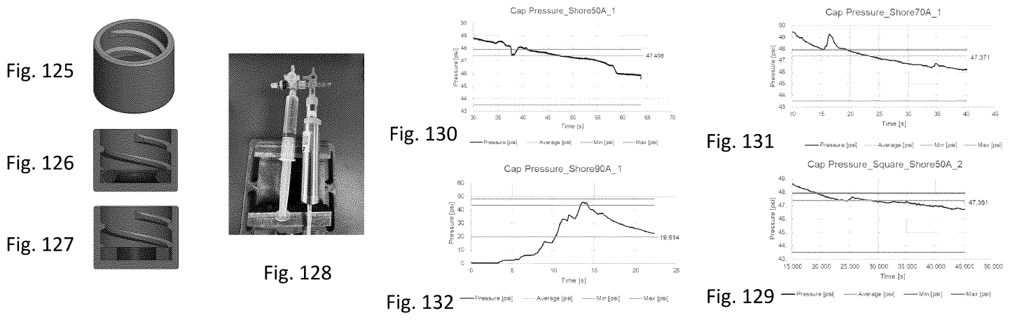

FIGS. 125-128 depict various views of a luer lock cap, representing the housing of the lamprey lock device 600b and a seal, such as an O-ring. FIG. 129 illustrates cap pressure testing of the luer lock cap using an O-ring seal having a shore hardness of 50A Square. FIG. 130 illustrates cap pressure testing of the luer lock cap using an O-ring seal having a shore hardness of 50A. FIG. 131 illustrates cap pressure testing of the luer lock cap using an O-ring seal having a shore hardness of 70A. FIG. 132 illustrates cap pressure testing of the luer lock cap using an O-ring seal having a shore hardness of 90A.

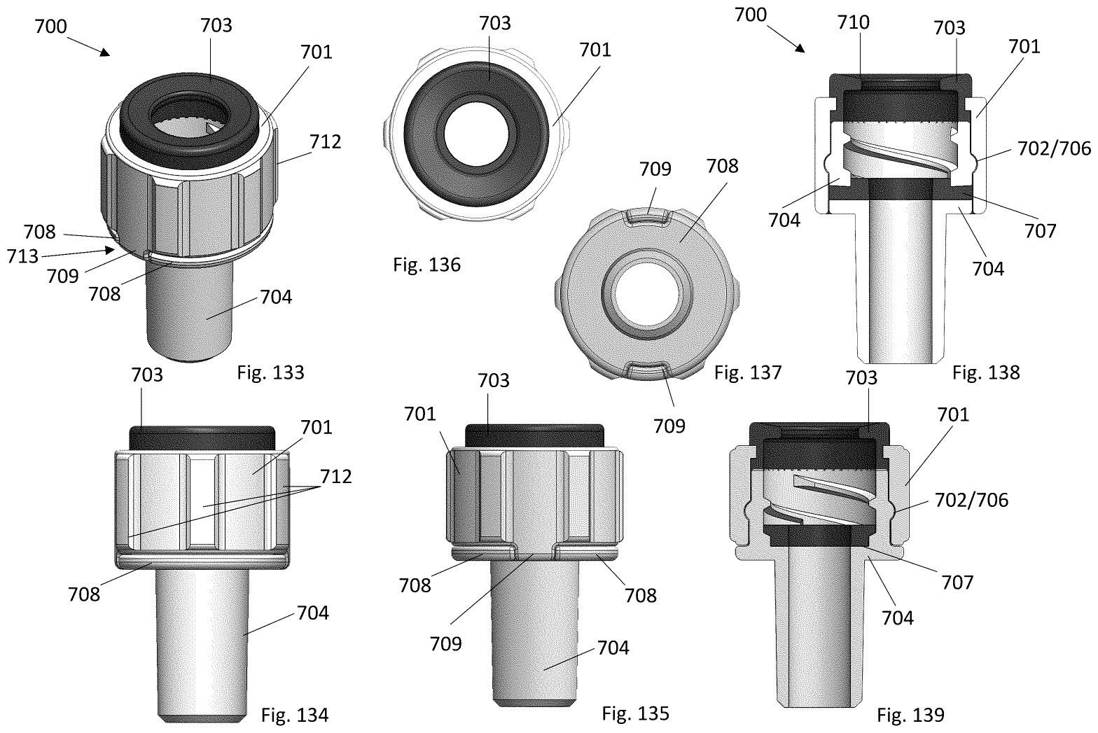

FIG. 133 depicts an isometric view of an exemplary lamprey lock device 700 having an anti-rotation feature and a grip feature. FIG. 134 depicts a side view of the lamprey lock device 700. FIG. 135 depicts another side view of the lamprey lock device 700 and the anti-rotation feature. FIG. 136 depicts a top view of the lamprey lock device 700. FIG. 137 depicts a bottom view of the lamprey lock device 700. FIGS. 138 and 139 depict cross-sectional views of the lamprey lock device 700.

The lamprey lock device 700 includes the same or similar features as lamprey lock device 600a and lamprey lock device 600b. For instance, lamprey lock device 700 includes a top portion 701 and a bottom portion 704 that are manufactured as two separate components and assembled and coupled to one another via one or more protrusions 706 positioned within one or more dents 702. Moreover, the lamprey lock device 700 includes an overmolded diaphragm 703 having a wiper 710 that includes the same or similar features as diaphragm 603a and diaphragm 603b and their respective wipers 610a and 610b.

The lamprey lock device 700 includes one or more gripping features 712 provided around the outer surface of the top portion 701. A gripping feature 712 may be a rigid body that protrudes from the outer surface of the top portion 701 to provide a textured surface for a user to grip. The lamprey lock device 700 may further include an anti-rotation feature that prevents the top portion 701 from rotating about the bottom portion 704. For example, the bottom portion 704 includes one or more recesses 713 within the bottom surface 708 of the bottom portion 704. The top portion 701 includes one or more protrusions 709 that extend from the bottom surface of the top portion 701. A protrusion 709 may be sized to reside within a recess 713, thereby interlocking the top portion 701 and bottom portion 704. The one or more protrusions 709 and recess 713 may form, for example, a dovetail interlock.

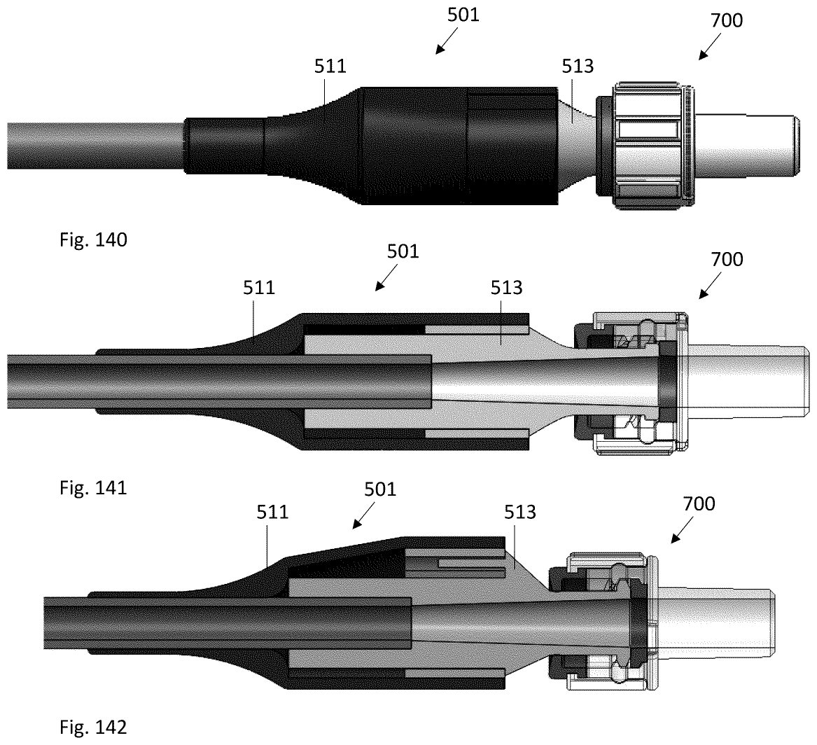

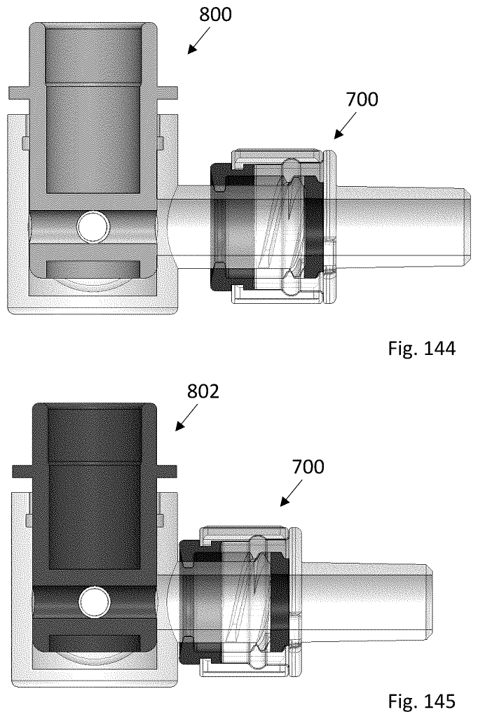

The lamprey lock device 700 may be coupled with a catheter 501, as illustrated in FIGS. 140-142, in a same or similar manner as the catheter 501 is coupled the lamprey lock devices 517, 520, 521, 540, 550, 560, 570, and 580 as described herein. Further, the lamprey lock device 700 may be coupled with either of luer stopcock 800 or luer stopcock 802, as illustrated in FIGS. 144 and 145. The lamprey lock device 700 provides a snug fit around a variety of catheters. Further, the one or more gripping features 712 of the lamprey lock device 700 allows a user to easily grip the lamprey lock device 700.

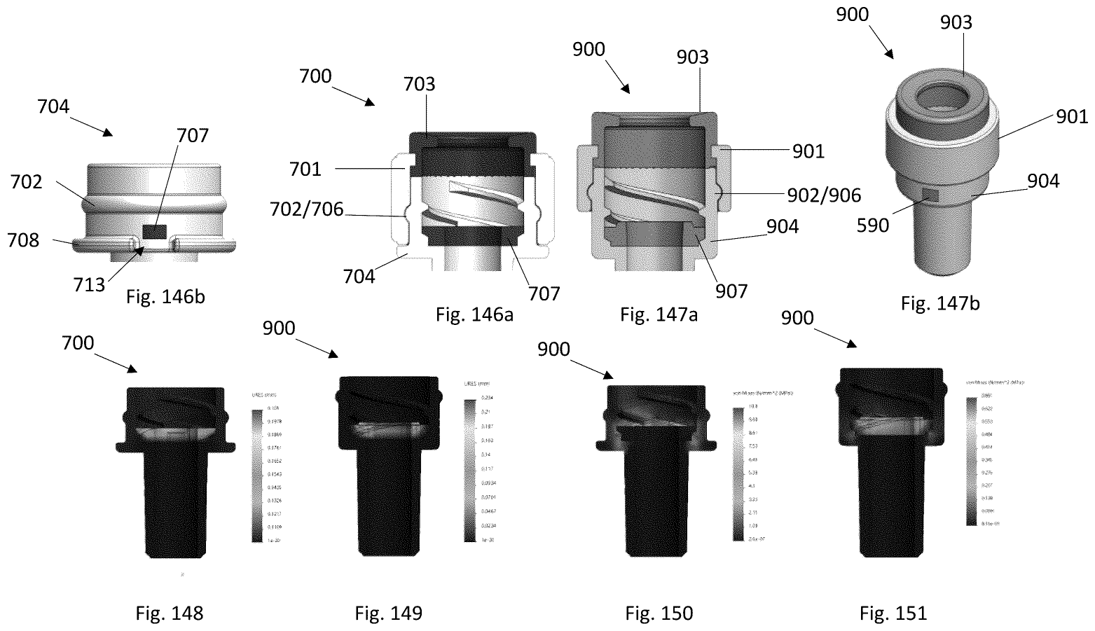

FIG. 146a illustrates a cross-sectional view of the lamprey lock device 700. FIG. 146b illustrates a side view of the bottom portion 704 of the lamprey lock device 700. The lamprey lock device 700 provides anti-rotation and grip features as described herein. The geometry of the bottom portion 704 provides a flat cross-section and a reduced overall height. The geometry of the top portion 701 provides a flat interface between components of the top portion 701 and bottom portion 704, thereby reducing the overall height of the lamprey lock device 700. The threads within the bottom portion 704 that interface with the luer fitting 513 of the catheter 501 provide about a 15% reduction in revolutions.

FIG. 147a illustrates a cross-sectional view of lamprey lock device 900. FIG. 147b illustrates an isometric view of the lamprey lock device 900. The lamprey lock device 900 includes the same or similar features as the lamprey lock device 700. For instance, the lamprey lock device 900 may be formed by a top portion 901 snapped onto a bottom portion 904 via one or more dents 902 and one or more protrusions 906. The bottom portion 904 may include a raised center. The top portion 901 may include a stepped interface between the bottom portion 904 and top portion 901. The luer fitting 513 may be seated in the threads of the bottom portion 904 within about 1.125 revolutions. The lamprey lock device 900 may provide a smooth exterior around the top portion 901. The lamprey lock device 900 may also provide that the top portion 901 freely rotates about the bottom portion 904. FIGS. 148-151 illustrate the unscrewing resistance for each of the lamprey lock device 700 and the lamprey lock device 900.

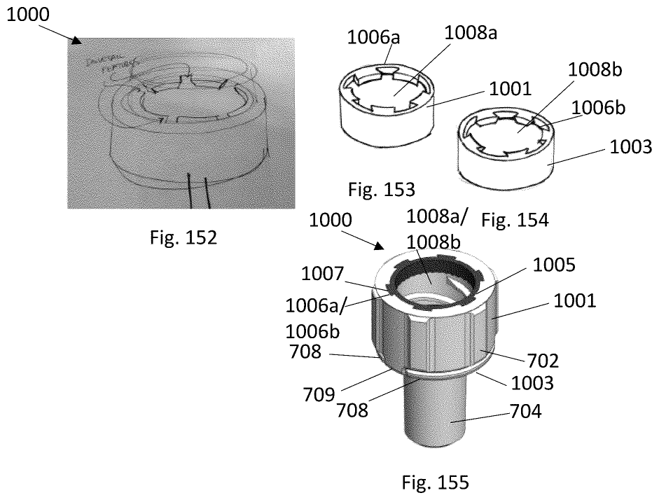

FIGS. 152-155 depict an exemplary lamprey lock device 1000. The lamprey lock device 1000 may include the same or similar features as lamprey lock device 700. The lamprey lock device 1000 may further include a dovetail interlock to couple the top portion 1001 with the bottom portion 1003. For example, the top portion 1001 and bottom portion 1003 may each have dovetail recesses 1006a and 1006b disposed around the openings 1008a and 1008b of top portion 1001 and bottom portion 1003. The top portion 1001 may be positioned on bottom portion 1003, such that the dovetail recesses 1006a and 1006b align with one another. A seal 1005 may be positioned in the openings 1008a and 1008b to interlock the top portion 1001 and bottom portion 1003. For example, the seal 1005 may include dovetail protrusions 1007 that correspond to the shape of the dovetail recesses 1006a and 1006b. The dovetail protrusions 1007 of the seal 1005 may be inserted into the dovetail recesses 1006a and 1006b, thereby interlocking the top portion 1001 and bottom portion 1003.

### Exemplary Non-Limiting Enumerated Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

What is claimed is:

1. A lamprey lock device comprising:
a first tubular shaped housing having a diaphragm on an end of the first housing; and
a second tubular shaped housing coupled to the first housing, wherein the second housing comprises a seal positioned on a bottom surface of a cavity defined by the second housing,
wherein the first housing, diaphragm, second housing, and seal define a channel to pass fluid through the lamprey lock device.

2. The device of claim 1, wherein an inner surface of the second housing comprises a threaded internal surface.

3. The device of claim 1, wherein the seal comprises an O-ring positioned on the bottom surface of the housing.

4. The device of claim 1, wherein the seal is overmolded to the bottom surface of the housing.

5. The device of claim 1, wherein the seal comprises a stepped surface provided on a proximal end of the seal.

6. The device of claim 1, wherein one of the first housing or second housing comprises one or more dents, the other of the first housing or second housing comprises one or more protrusions sized to interface with the one or more dents to couple the first housing and second housing to one another.

7. The device of claim 1, wherein the first housing comprises one or more dents on an inner surface that defines a cavity of the first housing, wherein the second housing comprises one or more protrusions on an outer surface of the second housing, and wherein the one or more protrusions are configured to interface with the one or more dents to interlock the first housing and second housing.

8. The device of claim 1, wherein the first housing comprises a plurality of gripping features a circumferentially disposed around an exterior surface of the first housing.

9. The device of claim 1, wherein the bottom surface of the second housing comprises a recess on along the perimeter of the bottom surface, wherein a second end of the first housing comprises a protrusion sized to interface with the recess of the second housing, and wherein the protrusion being positioned within the recess prevents the first housing from rotating about the second housing.

10. The device of claim 1, wherein the diaphragm comprises a wiper seal on the end of the first housing, wherein the wiper seal defines an opening to receive a luer fitting.

11. The device of claim 10, wherein the wiper seal is configured to flex inwards or outwards to provide a snug fit around the luer fitting.

12. A connection assembly, comprising:
a catheter having a luer fitting extending from a housing of the catheter; and
a lamprey lock device, comprising:
a first tubular shaped housing having a diaphragm on an end of the first housing, the diaphragm comprising a wiper seal that defines an opening to receive the luer fitting therein; and
a second tubular shaped housing coupled to the first housing, wherein the second housing comprises a seal positioned on a bottom surface of a cavity defined by the second housing,
wherein the first housing, diaphragm, second housing, and seal define a channel to pass fluid through the lamprey lock device, and
wherein the luer fitting and seal of the second housing interface with one another.

13. The connection assembly of claim 12, wherein the wiper seal is configured to flex inwards or outwards to provide a snug fit around the luer fitting.

14. The connection assembly of claim 12, wherein an inner surface of the second housing comprises a threaded internal surface that interfaces with an end of the luer fitting as the luer fitting is seated within the lamprey lock device.

15. The connection assembly of claim 12, wherein the seal comprises an O-ring positioned on the bottom surface of the housing.

16. The connection assembly of claim 12, wherein the seal is overmolded to the bottom surface of the housing.

17. The connection assembly of claim 12, wherein the seal comprises a stepped surface provided on a proximal end of the seal.

18. The connection assembly of claim 12, wherein one of the first housing or second housing comprises one or more dents, the other of the first housing or second housing comprises one or more protrusions sized to interface with the one or more dents to couple the first housing and second housing to one another.

19. The connection assembly of claim 12, wherein the first housing comprises a plurality of gripping features a circumferentially disposed around an exterior surface of the first housing.

20. The connection assembly of claim 12, wherein the bottom surface of the second housing comprises a recess on along the perimeter of the bottom surface, wherein a second end of the first housing comprises a protrusion sized to interface with the recess of the second housing, and wherein the protrusion being positioned within the recess prevents the first housing from rotating about the second housing.
